SEQUENCE LISTING (200 sequences)

SEQ ID NO: 1 - DNA, 18 bp, Artificial sequence
  Primer
gagagagagagagagaat

SEQ ID NO: 2 - DNA, 18 bp, Artificial sequence
  Primer
gagagagagagagagaaa

SEQ ID NO: 3 - DNA, 18 bp, Artificial sequence
  Primer
gagagagagagagagacg

SEQ ID NO: 4 - DNA, 18 bp, Artificial sequence
  Primer
gagagagagagagagatg

SEQ ID NO: 5 - DNA, 18 bp, Artificial sequence
  Primer
gagagagagagagagata

SEQ ID NO: 6 - DNA, 18 bp, Artificial sequence
  Primer
gagagagagagagagaca

SEQ ID NO: 7 - DNA, 19 bp, Artificial sequence
  Primer
cacacacacacacacaaac

SEQ ID NO: 8 - DNA, 19 bp, Artificial sequence
  Primer
cacacacacacacacaatg

SEQ ID NO: 9 - DNA, 19 bp, Artificial sequence
  Primer
cacacacacacacacaaga

SEQ ID NO: 10 - DNA, 19 bp, Artificial sequence
  Primer
cacacacacacacacaact

SEQ ID NO: 11 - DNA, 19 bp, Artificial sequence
  Primer
cacacacacacacacatag

SEQ ID NO: 12 - DNA, 19 bp, Artificial sequence
  Primer
cacacacacacacacattt

SEQ ID NO: 13 - DNA, 19 bp, Artificial sequence
  Primer
cacacacacacacacatgc

SEQ ID NO: 14 - DNA, 19 bp, Artificial sequence
  Primer
cacacacacacacacatca

SEQ ID NO: 15 - DNA, 19 bp, Artificial sequence
  Primer
cacacacacacacacagag

SEQ ID NO: 16 - DNA, 19 bp, Artificial sequence
  Primer
cacacacacacacacagtc

SEQ ID NO: 17 - DNA, 19 bp, Artificial sequence
  Primer
cacacacacacacacaggt

SEQ ID NO: 18 - DNA, 19 bp, Artificial sequence
  Primer
cacacacacacacacagca

SEQ ID NO: 19 - DNA, 19 bp, Artificial sequence
  Primer
cacacacacacacacactc

SEQ ID NO: 20 - DNA, 19 bp, Artificial sequence
  Primer
cacacacacacacacacga

SEQ ID NO: 21 - DNA, 19 bp, Artificial sequence
  Primer
cacacacacacacacacct

SEQ ID NO: 22 - DNA, 19 bp, Artificial sequence
  Primer
atatatatatatatattta

SEQ ID NO: 23 - DNA, 19 bp, Artificial sequence
  Primer
atatatatatatatattat

SEQ ID NO: 24 - DNA, 19 bp, Artificial sequence
  Primer
atatatatatatatattgg

SEQ ID NO: 25 - DNA, 19 bp, Artificial sequence
  Primer
atatatatatatatattca

SEQ ID NO: 26 - DNA, 19 bp, Artificial sequence
  Primer
atatatatatatatatggc

SEQ ID NO: 27 - DNA, 19 bp, Artificial sequence
  Primer
atatatatatatatatgta

SEQ ID NO: 28 - DNA, 19 bp, Artificial sequence
  Primer
atatatatatatatatgag

SEQ ID NO: 29 - DNA, 19 bp, Artificial sequence
  Primer
atatatatatatatatgct

SEQ ID NO: 30 - DNA, 19 bp, Artificial sequence
  Primer
atatatatatatatataac

SEQ ID NO: 31 - DNA, 19 bp, Artificial sequence
  Primer
atatatatatatatatacg

SEQ ID NO: 32 - DNA, 19 bp, Artificial sequence
  Primer
atatatatatatatatagg

SEQ ID NO: 33 - DNA, 19 bp, Artificial sequence
  Primer
atatatatatatatatcta

SEQ ID NO: 34 - DNA, 19 bp, Artificial sequence
  Primer
atatatatatatatatccg

SEQ ID NO: 35 - DNA, 19 bp, Artificial sequence
  Primer
atatatatatatatatcac

SEQ ID NO: 36 - DNA, 19 bp, Artificial sequence
  Primer
atatatatatatatatcgt

SEQ ID NO: 37 - DNA, 19 bp, Artificial sequence
  Primer
ctctctctctctctctaat

SEQ ID NO: 38 - DNA, 19 bp, Artificial sequence
  Primer
ctctctctctctctctata

SEQ ID NO: 39 - DNA, 19 bp, Artificial sequence
  Primer
ctctctctctctctctacg

SEQ ID NO: 40 - DNA, 19 bp, Artificial sequence
  Primer
ctctctctctctctctagc

SEQ ID NO: 41 - DNA, 19 bp, Artificial sequence
  Primer
ctctctctctctctcttaa

SEQ ID NO: 42 - DNA, 19 bp, Artificial sequence
  Primer
ctctctctctctctctttg

SEQ ID NO: 43 - DNA, 19 bp, Artificial sequence
  Primer
ctctctctctctctcttct

SEQ ID NO: 44 - DNA, 19 bp, Artificial sequence
  Primer
ctctctctctctctcttgg

SEQ ID NO: 45 - DNA, 19 bp, Artificial sequence
  Primer
ctctctctctctctctcag

SEQ ID NO: 46 - DNA, 19 bp, Artificial sequence
  Primer
ctctctctctctctctcct

SEQ ID NO: 47 - DNA, 19 bp, Artificial sequence
  Primer
ctctctctctctctctcgg

SEQ ID NO: 48 - DNA, 19 bp, Artificial sequence
  Primer
ctctctctctctctctgac

SEQ ID NO: 49 - DNA, 19 bp, Artificial sequence
  Primer
ctctctctctctctctgtg

SEQ ID NO: 50 - DNA, 19 bp, Artificial sequence
  Primer
ctctctctctctctctgct

SEQ ID NO: 51 - DNA, 19 bp, Artificial sequence
  Primer
ctctctctctctctctggc

SEQ ID NO: 52 - DNA, 19 bp, Artificial sequence
  Primer
ctctctctctctctctaaa

SEQ ID NO: 53 - DNA, 19 bp, Artificial sequence
  Primer
ctctctctctctctctaac

SEQ ID NO: 54 - DNA, 19 bp, Artificial sequence
  Primer
ctctctctctctctctaag

SEQ ID NO: 55 - DNA, 19 bp, Artificial sequence
  Primer
ctctctctctctctctatt

SEQ ID NO: 56 - DNA, 19 bp, Artificial sequence
  Primer
ctctctctctctctctatc

SEQ ID NO: 57 - DNA, 19 bp, Artificial sequence
  Primer
ctctctctctctctctatg

SEQ ID NO: 58 - DNA, 19 bp, Artificial sequence
  Primer
ctctctctctctctctaca

SEQ ID NO: 59 - DNA, 19 bp, Artificial sequence
  Primer
ctctctctctctctctact

SEQ ID NO: 60 - DNA, 19 bp, Artificial sequence
  Primer
ctctctctctctctctacc

SEQ ID NO: 61 - DNA, 19 bp, Artificial sequence
  Primer
ctctctctctctctctaga

SEQ ID NO: 62 - DNA, 19 bp, Artificial sequence
  Primer
ctctctctctctctctagt

SEQ ID NO: 63 - DNA, 19 bp, Artificial sequence
  Primer
ctctctctctctctctagg

SEQ ID NO: 64 - DNA, 19 bp, Artificial sequence
  Primer
ctctctctctctctcttat

SEQ ID NO: 65 - DNA, 19 bp, Artificial sequence
  Primer
ctctctctctctctcttac

SEQ ID NO: 66 - DNA, 19 bp, Artificial sequence
  Primer
ctctctctctctctcttag

SEQ ID NO: 67 - DNA, 19 bp, Artificial sequence
  Primer
ctctctctctctctcttta

SEQ ID NO: 68 - DNA, 19 bp, Artificial sequence
  Primer
ctctctctctctctctttt

SEQ ID NO: 69 - DNA, 19 bp, Artificial sequence
  Primer
ctctctctctctctctttc

SEQ ID NO: 70 - DNA, 19 bp, Artificial sequence
  Primer
ctctctctctctctcttca

SEQ ID NO: 71 - DNA, 19 bp, Artificial sequence
  Primer
ctctctctctctctcttcc

SEQ ID NO: 72 - DNA, 19 bp, Artificial sequence
  Primer
ctctctctctctctcttcg

SEQ ID NO: 73 - DNA, 19 bp, Artificial sequence
  Primer
ctctctctctctctcttga

SEQ ID NO: 74 - DNA, 19 bp, Artificial sequence
  Primer
ctctctctctctctcttgt

SEQ ID NO: 75 - DNA, 19 bp, Artificial sequence
  Primer
ctctctctctctctcttgc

SEQ ID NO: 76 - DNA, 19 bp, Artificial sequence
  Primer
ctctctctctctctctcaa

SEQ ID NO: 77 - DNA, 19 bp, Artificial sequence
  Primer
ctctctctctctctctcat

SEQ ID NO: 78 - DNA, 19 bp, Artificial sequence
  Primer
ctctctctctctctctcac

SEQ ID NO: 79 - DNA, 19 bp, Artificial sequence
  Primer
ctctctctctctctctctt

SEQ ID NO: 80 - DNA, 19 bp, Artificial sequence
  Primer
ctctctctctctctctcta

SEQ ID NO: 81 - DNA, 19 bp, Artificial sequence
  Primer
ctctctctctctctctctc

SEQ ID NO: 82 - DNA, 19 bp, Artificial sequence
  Primer
ctctctctctctctctctg

SEQ ID NO: 83 - DNA, 19 bp, Artificial sequence
  Primer
ctctctctctctctctcca

SEQ ID NO: 84 - DNA, 19 bp, Artificial sequence
  Primer
ctctctctctctctctccc

SEQ ID NO: 85 - DNA, 19 bp, Artificial sequence
  Primer
ctctctctctctctctccg

SEQ ID NO: 86 - DNA, 19 bp, Artificial sequence
  Primer
ctctctctctctctctcga

SEQ ID NO: 87 - DNA, 19 bp, Artificial sequence
  Primer
ctctctctctctctctcgt

SEQ ID NO: 88 - DNA, 19 bp, Artificial sequence
  Primer
ctctctctctctctctcgc

SEQ ID NO: 89 - DNA, 19 bp, Artificial sequence
  Primer
ctctctctctctctctgaa

SEQ ID NO: 90 - DNA, 19 bp, Artificial sequence
  Primer
ctctctctctctctctgat

SEQ ID NO: 91 - DNA, 19 bp, Artificial sequence
  Primer
ctctctctctctctctgag

SEQ ID NO: 92 - DNA, 19 bp, Artificial sequence
  Primer
ctctctctctctctctgta

SEQ ID NO: 93 - DNA, 19 bp, Artificial sequence
  Primer
ctctctctctctctctgtt

SEQ ID NO: 94 - DNA, 19 bp, Artificial sequence
  Primer
ctctctctctctctctgtc

SEQ ID NO: 95 - DNA, 19 bp, Artificial sequence
  Primer
ctctctctctctctctgca

SEQ ID NO: 96 - DNA, 19 bp, Artificial sequence
  Primer
ctctctctctctctctgcc

SEQ ID NO: 97 - DNA, 19 bp, Artificial sequence
  Primer
ctctctctctctctctgcg

SEQ ID NO: 98 - DNA, 19 bp, Artificial sequence
  Primer
ctctctctctctctctgga

SEQ ID NO: 99 - DNA, 19 bp, Artificial sequence
  Primer
ctctctctctctctctggt

SEQ ID NO: 100 - DNA, 19 bp, Artificial sequence
  Primer
ctctctctctctctctggg

SEQ ID NO: 101 - DNA, 19 bp, Artificial sequence
  Primer
atatatatatatatatttc

SEQ ID NO: 102 - DNA, 19 bp, Artificial sequence
  Primer
atatatatatatatatttg

SEQ ID NO: 103 - DNA, 19 bp, Artificial sequence
  Primer
atatatatatatatatttt

SEQ ID NO: 104 - DNA, 19 bp, Artificial sequence
  Primer
atatatatatatatattag

SEQ ID NO: 105 - DNA, 19 bp, Artificial sequence
  Primer
atatatatatatatattaa

SEQ ID NO: 106 - DNA, 19 bp, Artificial sequence
  Primer
atatatatatatatattac

SEQ ID NO: 107 - DNA, 19 bp, Artificial sequence
  Primer
atatatatatatatattga

SEQ ID NO: 108 - DNA, 19 bp, Artificial sequence
  Primer
atatatatatatatattgt

SEQ ID NO: 109 - DNA, 19 bp, Artificial sequence
  Primer
atatatatatatatattgc

SEQ ID NO: 110 - DNA, 19 bp, Artificial sequence
  Primer
atatatatatatatattct

SEQ ID NO: 111 - DNA, 19 bp, Artificial sequence
  Primer
atatatatatatatattcc

SEQ ID NO: 112 - DNA, 19 bp, Artificial sequence
  Primer
atatatatatatatattcg

SEQ ID NO: 113 - DNA, 19 bp, Artificial sequence
  Primer
atatatatatatatatggg

SEQ ID NO: 114 - DNA, 19 bp, Artificial sequence
  Primer
atatatatatatatatgga

SEQ ID NO: 115 - DNA, 19 bp, Artificial sequence
  Primer
atatatatatatatatggt

SEQ ID NO: 116 - DNA, 19 bp, Artificial sequence
  Primer
atatatatatatatatgtg

SEQ ID NO: 117 - DNA, 19 bp, Artificial sequence
  Primer
atatatatatatatatgtt

SEQ ID NO: 118 - DNA, 19 bp, Artificial sequence
  Primer
atatatatatatatatgtc

SEQ ID NO: 119 - DNA, 19 bp, Artificial sequence
  Primer
atatatatatatatatgaa

SEQ ID NO: 120 - DNA, 19 bp, Artificial sequence
  Primer
atatatatatatatatgac

SEQ ID NO: 121 - DNA, 19 bp, Artificial sequence
  Primer
atatatatatatatatgat

SEQ ID NO: 122 - DNA, 19 bp, Artificial sequence
  Primer
atatatatatatatatgca

SEQ ID NO: 123 - DNA, 19 bp, Artificial sequence
  Primer
atatatatatatatatgcc

SEQ ID NO: 124 - DNA, 19 bp, Artificial sequence
  Primer
atatatatatatatatgcg

SEQ ID NO: 125 - DNA, 19 bp, Artificial sequence
  Primer
atatatatatatatatata

SEQ ID NO: 126 - DNA, 19 bp, Artificial sequence
  Primer
atatatatatatatatatt

SEQ ID NO: 127 - DNA, 19 bp, Artificial sequence
  Primer
atatatatatatatatatg

SEQ ID NO: 128 - DNA, 19 bp, Artificial sequence
  Primer
atatatatatatatataaa

SEQ ID NO: 129 - DNA, 19 bp, Artificial sequence
  Primer
atatatatatatatataag

SEQ ID NO: 130 - DNA, 19 bp, Artificial sequence
  Primer
atatatatatatatataat

SEQ ID NO: 131 - DNA, 19 bp, Artificial sequence
  Primer
atatatatatatatataca

SEQ ID NO: 132 - DNA, 19 bp, Artificial sequence
  Primer
atatatatatatatatacc

SEQ ID NO: 133 - DNA, 19 bp, Artificial sequence
  Primer
atatatatatatatatact

SEQ ID NO: 134 - DNA, 19 bp, Artificial sequence
  Primer
atatatatatatatataga

SEQ ID NO: 135 - DNA, 19 bp, Artificial sequence
  Primer
atatatatatatatatagc

SEQ ID NO: 136 - DNA, 19 bp, Artificial sequence
  Primer
atatatatatatatatagt

SEQ ID NO: 137 - DNA, 19 bp, Artificial sequence
  Primer
atatatatatatatatctg

SEQ ID NO: 138 - DNA, 19 bp, Artificial sequence
  Primer
atatatatatatatatctc

SEQ ID NO: 139 - DNA, 19 bp, Artificial sequence
  Primer
atatatatatatatatctt

SEQ ID NO: 140 - DNA, 19 bp, Artificial sequence
  Primer
atatatatatatatatccc

SEQ ID NO: 141 - DNA, 19 bp, Artificial sequence
  Primer
atatatatatatatatcct

SEQ ID NO: 142 - DNA, 19 bp, Artificial sequence
  Primer
atatatatatatatatcca

SEQ ID NO: 143 - DNA, 19 bp, Artificial sequence
  Primer
atatatatatatatatcaa

SEQ ID NO: 144 - DNA, 19 bp, Artificial sequence
  Primer
atatatatatatatatcat

SEQ ID NO: 145 - DNA, 19 bp, Artificial sequence
  Primer
atatatatatatatatcag

SEQ ID NO: 146 - DNA, 19 bp, Artificial sequence
  Primer
atatatatatatatatcgg

SEQ ID NO: 147 - DNA, 19 bp, Artificial sequence
  Primer
atatatatatatatatcga

SEQ ID NO: 148 - DNA, 19 bp, Artificial sequence
  Primer
atatatatatatatatcgc

SEQ ID NO: 149 - DNA, 768 bp, Pongamia pinnata
aggggagaactccatttgcgaaggagtagattatttccttagataagaaatccggttcgtggcacaataaaacaaaggtttaaataaaaatttatcgttattagactcgtaacttaccttcttcatggctcaacccaatctgtatatcgacttatttaatttggttaacaaatcggttttcgtcgcaaataggggatgacaactaaaataccaatttgtgtccttgcgacacgtcaacaggtggctgccaatatctcaagttttgagcacacaacgctgtgattggctatctgtagatcgcttcaagaactttcggaccgacatatcacaacacaatcatgtccaatccaactaacgattggtcgaaaccgcacttgatcgcttcatttcaaccaattcccatctactaaaacgagcgtcggaataatctcgaacgtaaccccacgtggtgggtggcagcgcccctcgtatctttcaatcgccactcgagtctcaagtttgctcactttgaaattttccatctggactacacatcagatcaaactcaacttttatcaattgaaacacaagggttcatctcaatgctgccacttcttctcgtcgtgaccatcgcatgaggcggtggcaaccacaaatctgtgtcccggtcgatgcagccaatgtgaacttggccaggagctgttaaccggagggtgggtgccccttcctgatcgaagtggaggggctatgtcaccggccgcaggtttagggtagtcaggaaaagggatg

SEQ ID NO: 150 - DNA, 219 bp, Pongamia pinnata
cggttagatccggtcagtccttctccgccccggcaccggcggcacctcgactccgggccacctctgcaaaagacaaattgaaggagaatccccttaaaacaggcaggtgagacaccctcagattcgctctacccccccactcaaccccaaaaactcaccttcaatacagcccatattaaccacccctgtcaatgtcccccccatcctcccggatccaat

SEQ ID NO: 151 - DNA, 232 bp, Pongamia pinnata
agttaaattccatgatgagtgaactcagacgccccgacccccacggaactcggtcctggatacgctggattgaaggaaaaaaaggtttcccctgcttgctggtctggtgacaccttcagattcgccccacctcacccggctaaacgctttggtccgaacttttcccataactgttgtaaaattgatgctgcaataacgaaacccgtcccagccaggcctcgcagtcttaccc

SEQ ID NO: 152 - DNA, 503 bp, Pongamia pinnata
tatgaattctctttaagctaatgcttcagcttctacttcctttctttgtctggatgatctcccctcatctatgaaaaaacaattggggagagaggaattcgtgcatgatggacgttttacccccctgcccctctccacaaatattaagcattgcatttctaatggatagaaaattactacatactttttttgtcactcgtgtttgaagtgccctttatcccggtttctcgaacaattaaactttactttaatgaaaatagaatccacgctaggagagttcaaccttggtacctccattgattggacggggggggggggggggggtgggggggtggtcagtagggggaaacaagagttggtcgcccttctccttggtgtcattgctgatgacggactgggagcaaggttgcatccatcgggaaacttacggggttcattagttgtcgatttcctacaagggatgaagggcaccttcccatcgagttcacagtgttcctaggcat

SEQ ID NO: 153 - DNA, 1089 bp, Pongamia pinnata
taggaatacgagatgagtgaatgctccatctagatgctgcacaagccggctagtgtgagtggatggatatctgcttaattaggctgctttggcctgtgagagaaaaaagaaaaaacaagcaagcaacaaccctatctatccttccctgcctgcctgccatggccccaccctgcctgcgctaatctcatctgggcagagcatctctttgctttgctcccatctaatctcccctaaggcaaatttaattccctggtctggcggccattggatccgaattcgatctcggtatcggccttggatggtcatggtgttttttgtgtcaaattgtaatttgctctccgctccccattccatacaacccacaagccaaagggaaaagccgggaatgccgaggggccgaatgagtgaacttaattgattaaacgcactgccctctttgccctctttaaacccgggaaacctgctgcattaatgagccttaatgaatccgccgacacgcggtttgagtattgggcggtattggcttcccttccccttgactccctgctgactctcggtgcttgctccttccggtgccaacacaggtatcagatcaatccggttatctactaaagttagtgataaagcaagaatgatcatgtaagcaaaaagctgtgaaacggccagcaaccaaaagaatgccgaatcgtataaagttcttccattcggtcgtcccgcccgtaagagcctccccccatcgaagctctcactaaaatgcgacgctccccttcagagagtgagcgagaccccacacgtttctcatgataccacccgcttccgctctgctgtctcactctggcgctcaccagataccgatcctgctgctacctgatacatgtcggccctttctccttctagaagcgtggtatctatctctcagtgtacggtgtagctccacacgtgcctgtgtggtcattcccctgttaaccggactgttggccttatcacgccagtacagcctgacgtccgtcccgttacacgttactatcgcctgagttctaccctgtgagacagactatctataacccagtggatatcagctcctgcgta

SEQ ID NO: 154 - DNA, 481 bp, Pongamia pinnata
ggccatacaatgaatacgccaagctggaagtctcgcccgatctgtactggctcactattctcatttaaattttcagagcgcaaaaatggctgaaatcactcacaacgatggaaactctaacaacttggaaatgaaataagcttgcatgtcaggctggaaggcacataatgatttttattttgactgatagtgacctgttcgttgcaacaaattgatcagcaatgctttcttataatgccaactttgtacaacaaagctgggtgggccctctcgatctgcatgcctagacattctctaatgaaaaaatctttcagtcgaaaatagaaaatgagttaaagttggagtttttattgaaaacagacttccgtgtggattagtgtttttagcgagtgtgacaggacagcaaaaaaatacataatcaaggggggaactgaaaacttaggaatgcatataactacccaggagaacaagacttccccga

SEQ ID NO: 155 - DNA, 185 bp, Pongamia pinnata
tattaaaacattaattcgttctacctgccgaccacgaagggaactcgtcttttagacactctggtatatttcgaaaagaaggacagcgtggttatcaagggacggaaaccccccttgatgcctcccacctccccttcttttactctacaacatcgtcttgaatcacttcccccgcccgccaacgc

SEQ ID NO: 156 - DNA, 189 bp, Pongamia pinnata
tcaatttgcattgaatgcagtcaagcccgtcgggccccaccgggactcgtctttttggccaactctggtaaatttcagaagagaggagagactgtgaaaatcgaaagatgggaaatctccccttaaaccgcccacccccaccccaaagacttcgcaacacctactattatgttgttcgtggccattaac

SEQ ID NO: 157 - DNA, 770 bp, Pongamia pinnata
taacgctgcctgattagttaccgagacatgaacttcgctcctccgctgtgagctgggcctggtcgtgtcccccccgccgaaacgagaagggacgagcaggacagcccagaacgctcacgtttttggctatttcggtcccgtcgggttgactgggatgttatatgttgtgtctttacaatgagtctgagatgtcggctgacgatgccctaccccggttgtaaagatacctaccatgaatacgtcggatggtccaaacgactctggaatataggcaccaggtctctactgttttatcaggtgaaagtgccagtgttgtccattttttttgcgaaaacttcctgggcgaggtgtctcctccattttatttttaactccatttgggtttcaggatcaaggttgcaaaaaaacatttttttaactaaccctattttattaaattttagcctaaaataccaaggtggcgacggtattttagtttttttaattattaggggagttagaacaaacaaaggatagggtgactacatgacggggacaaattcgtaaatcagagaaaataaagagagagacgttatcttgtaaagagtactaaacgccagcctgtgtaatcagactacgactcttcggaggagatcaacggagaaaagcgtcgcgtcgggaaagcaagcagagggacggcgtctctgacgctgcctctggacacctctagagagtcccgctccacagtggaatgcaccccggcggctccctagattgaatgggagaaacga

SEQ ID NO: 158 - DNA, 1027 bp, Pongamia pinnata
tcaggtgtggtggaggggattattgactttagtgctgatggaccagccgctagtgggatgcgagaattacagcgttgtgcgtctttctttggccgaccgtgacaagaatgaataatagtctagtcagcatccttgtcaacccttgctgccctgccccatcatgggggttccctgcggaatgctggtcactcctgcctttacaacctctttgccgccatcccccctactctccccactgccgcattcctctcactgggggctcttaattttgattcaaaactgctcggtaactggcccgaatcattcggaggcctgaccggtgcgtgagattgttttctgtccgcatttcgatccaccatacatgccagacgcattaattgtaaagccgcggggggtgctgaaagagcggatcacattgattgcgttgcgctgactgcccgctttccattccaaaaacctgtcttgccaggcccattgcagtaacgaccaacccgttgagagaggagaggcgcgtattgggcgctcttcctcttcctctctcactcactctctgcgctctgtctttcgcctgcggctaccggtatccactcactccaaggcgatagtacggtaatccgttaatcaccgagaaacggcaagaaagaacgagtgaccatgaggccaaaaagccacaaaaacgccaggaacgaccggaagggcgcgcttttttggattgttcgctagcgctgagacccctcgcacaaaccactctcaaggcacagttggttgagagggagcgatactgacgagacagagattttcccctgcatctccctctggagctatcctgttacactctgccgttagaggatgcgtgttgccttattccttccccatttgtatctctttgtatattggcgctatatgatctcacatctgaatgctgattgctcattgcagtctgtgtgactaacacgagtttaagcacaaaactctcgtaagcactgaatgtgctgtctatccgatgcagtgaataacttacttttgc

SEQ ID NO: 159 - DNA, 1054 bp, Pongamia pinnata
caaaataaataggggggaaaaagtggccctccaagatccaagctccagcggcctcttgtgtgagggatatctgcataattcgtttttccagctttggcctgtgacaacaagaaacaaaaatcaagcaagcagcatccctatcaagccttgccaccctgccccatcatggccccaccctgtcaactgctaatctctcctgggcatacatgctctttgccttcatcccagctaatctccccacaaggcaaattccggcagactggtttccgttacaagtggatacgaactcggttccgttatggacgtaatcttggtcataactgtttcttgtgattaattgttatcctctcacaattcctcactaccaacaatttgaagaataaagtgaaagcctggggtgcctaatgagggacctaactcatttaattgcgatgcgctcactgcccactttttttcgggaaacctgtcttgccaactgcattaatgaaacggccaacgcgcggggacaggcggtttgcgtattgggcgctcttccgcttcctcgctcactgactcccttctctttttctttcggctgctcctagcgttatcaacacactcaaaggcagatatactgttatcttcaaaatcttgttataacgcacgaatgaacatgtgagcaaatagctatcagtaggccagcaaccgtagaaatgccgcgtcttctaggattgttcaattcgctccgaccgcctggaagagcatccataactcgacgctcagttcatatgtgactataccgacaggactaaaagaacccattcgtttcccttgatatctccctcgtgcgctctcctgtaccaaccctgcatttaccagatacctgtccacctatctctcttcgggaatcttggcgctttctctttctcacactgtgagtatcagatttcagtctacttggtcgctcaaacgggctgtgagtgttttctcatgttagacagactgctgggcttaccggaagctaccgctttattctacccgtaaaacttacaatacgcactgggtttattaatgg

SEQ ID NO: 160 - DNA, 279 bp, Pongamia pinnata
aatagaaaagggtaatacgccaaactgctcgaggcacagctggaacttactgggttggatacgcttggattttcaccatgaaaagggagggggtgtgataaaggagaggacggtaactcccgtttcatgttccgacccgcccttcactgtcctgcctacaccagtgcgtatgatcaacttataactcatattaacccgtccctcccccacaaaagtttatcaacaaatctccccacccccccctttataatgtccaggtggttatcctctcacgtaata

SEQ ID NO: 161 - DNA, 318 bp, Pongamia pinnata
tgaagccataaatgaattacccgaggctggtatggggcgcaagagcttactagctttttaatctcatttaattttaagaaagaagaaagagtgagtcagtcgaacgatggaaccctcgcccttaggaatgaaattccttgcattgcgcagaaatcccacaatgattttattttgactgatagtgacctgttctccccaacaaattgataaacaatgctttcttagaatgccaactttgtacaagaaagctgggtgggccctctagaactgcttgcttaacattctctaaagaaaaatctttcagtggaggttgaggag

SEQ ID NO: 162 - DNA, 623 bp, Pongamia pinnata
cacacctgtcaccttgacaagagggatcaaaacacatcggtactttccgccaactgaacgaggtaggctctcactgcgtcgagaaaaagggagaaaagggctgtgaagacgaagagcaccttgagccccacccccacctctcgctggccgtctgcattactatcggcctctgtcatatacttgatcacggctattagggcaatgttgggaatacacgacttaaagcaacatattcacttcgctataagtgaaccagacaacctcttaaaacagacaacatatcctactacttccaaacaagtggtagaacgtaccacctgcttgttctcacacgttaaagttgtctccgcccgactcgacaattttcatttatttttcacgaagttctctcagtttaaaaagcatgcgcgactagctttgcttgttttcgcttatctaaaagcacaaccatatttggcccacggcccaagctgtataacaaacattatattgctagataaacttccgtccgcttaagaaggcctcattccccaaaataatactccattgggatgttgtgtagtgtgccgatacatcccatctagcaggctcaagatttccctaaaggcacatcgaccccccat

SEQ ID NO: 163 - DNA, 872 bp, Pongamia pinnata
gcaccctggagggttaaattaacgccccctattcatcggtgaatcatcttcggaacgaaggtgcccagcgctacagcagggggaggggatgaaacggcgtgcagggcgacggagtatcaagagcgagacccgcccctctcgctgagactctgcccgacacctcggccttggagatctacatggtgcatgctatccagagcaaagttgaagagacacccgaacagaagcgatagtccttttcaaacttcccagtgaaccagctgaaatccgcaatactaccaccataactttgttctttagcaccatcctgttggacaacaccacctgcttgaaaggacacgtgaatgatgtccaggtgagggtctacaattttaggttttgttaaccacgtgttctcattttttaagcgtgagcctatgtgactaattgccgcgccgtctgtcagcacgtcctttttgggctcgcggccaagtcttcgatactacatattcctgctgatcaactttaggaagctaacaactctcaattcccccaacaaaaccctccttttatgattattatttagtacaacaaagctcatatgcaatgcttaagaatgcctgaaaataacaccaagaccccttagtagagccttttcctaaggaaacattttttcatgtttgagtaattatcgccaacctgttcaacgcactacaacgactctaatcgataccgacaggagaaccaggttgatcgagtcgggacaaagatgagacgtcagggacatcatctgtgacttctccggggaactactctaagatttcgctctccagttgggacatcgctccaccgacccgcatcacaaaattgacgcttagagaggcaaagatctc

SEQ ID NO: 164 - DNA, 565 bp, Pongamia pinnata
taacactttcgccttttcgtgtagctgaacacttctccgcgagctgacaccggggcgtggtcggggctcacccgccagaaacgacaggaaccagagggagaccaacaaagcctttcttttgacctttcgccaccccggaggggttggccgcctacgttccgaggcttattcataccactgattgaaacgtaccccgctggcgttgcaaaaatccccgttagagcaccacacaaccgaaaacggcggagggacgatgtgtccttgaacaataggctccacatatttacgactttacaggggaaatacccgtttgtgaaaatgtatatcgatagaattccttagccaggacatccacggccttcacaaccctgcagctgcctggaacaaggtataatgcaaaaaaactttagattaaaattcttatcagagaaagaagcagctcggaataacgggagacgaacttcgttactaaaataagctcaaaaacaatccaaagtaaaaaacaaagggagatacaaaacagggaaaacactttatcagattttccacctttgaagctatcacc

SEQ ID NO: 165 - DNA, 955 bp, Pongamia pinnata
tccataacaaaagagggatgatagtagtgacatcacgcgcgctcacacgcccgtatgccggaaggaaaaatggctcagcaattaagcctgaatgacctctttgttgagagatggatctttgcgagcttgatcattctcgcccatgtttcccgcctgggttgattgttatccacatctcgatgtgcgttctgggcggaaggtaccttgtattgttcggcccgcctttgaatgaactaatgtgtgtacattggttgcgctcactgcccggtctcgtgtcgggacactggtcgaggtaacttgataaatgaatccgccaaggcccacgctgttgcagaggccgtttgcgtatcgcttcctcttcctcttccacacacacagtgcccccgcgctggttggttcaactgcgtcgaccggttgcatctcagtcataggacgtattacggttaatcattgagattggccagctgcattagagtaaccatgaacccataaagacagcagaaaggtgtgttccgtcacattgcttactcttttccgattcccatccgccccaccctccctgcgcagcatgatcacaaatcaactgatcaggcgcaaaggtgacgaaaccccatgagactatacatataccagctcatactcccttgcaagcgcccttcatgaactctcgagtttctaacaaagcccagttaccggatatgttctccagcatttctcccttctggagatgtctcgcgaatacatatctcatccagagaggagcactcgtcagagtaggtacctagcacacaactgtcgcctttatgtacaaacgctacgttctgcccgaccgttgcacgtgtatgagacatgtgagcatcttagagtccgcgaaggtaacactactttaatctcatgtgaagagtctcgaatgcaatcaatggtagtagaagctaggtgtgtaggctgtgttaca

SEQ ID NO: 166 - DNA, 1213 bp, Pongamia pinnata
tcctaccgatcggggcgattgtacttaccggccgcggattcgcccttattactgtctgaataatggccaattcgtttaaccctgcgcgactactccctttactgagggttaattctgagcttgccgtaatcatggtcatagctgtttcctgtgtgaaattgttatcccctcgcaattccccacaacatacgagccggaagcataaagtgtaaagcctggggtgcctaatgagtgagctaactcacattagttgggttgcgctcactgccggctttccggtcgggaaacctgtcgtgctaactgcattaatgaatctgccaaggcgcggggagaggcggtttgcgtatcgggcgctcttccgcttcctcgctcaatgactcactgcgctcggtgcgttcggctgcagcaatcggtatcaggttactctgaggcggtagtacggttatccgtagaatcacgggataagacaggagcaaacatgtgatcaaaaagccgcggaaaggcgaggaacccgaaaagggccccgttgctggtgtttttccatagggctccctccgctcggacgctatcccgcggctcgacgctcaagtccaatgtggggaaagccgacaggactataaagaatctaggcatttccccctggaagctccctcgtgcgctgtcctgttccgaccctgacccgtacctaagacccgttacgccgttcttccttcggcaatttccccgctttctgatatttcgcatatatgtacccaattcggggggaggtaattcgttcggagtatggctgagcacgcgattcccccgtaatctcccagcgctggcccttctgcactaaccattacccttgaggccagacctgtaagcactttagcgtttcgccaaagcgtgccaccattttaaacagcattatgtggagcgaagcatggcaggcggaggtagtcgtttctaggaaatgtgggcgtactgccgccccatctaaccgaacgtcattggtatctggcttcctaaatcagatttcttttccagaaaaaattttggttactgtagttccgttcacaaaccgcattaatgggtatcgattttatgtgttttgtgacggaggcacccaaatttctgcattaatgaggcccttatcaacgcaaatcccagaaaaaatataataaatgtggggaaggacccctccgtcgaagcccaaaaacatgcttgataa

SEQ ID NO: 167 - DNA, 478 bp, Pongamia pinnata
ccaccattgacttgcattacgccaagctggagtttcgcccgctctgtactgactaactattctcatttaaattttcattagcttaaaaatggctgaaatcactcacaacgatggaaactctaacaacttggaaatgaaataagcttgcatgcaggctggaaggcaaataatgattttattttgactgatagtgacctgttcgttgcaacaaattgataagcaatgctttcttataatgccaactttgtacaacaaagctgggtgggccctctagatctgcatgcctagacattctctaatgaaaaaatctttcagtgaaaagtgaacatgagttaaagttggagtttttattgaaaacagatttccgtgtgattagtgtttttagcgagtgtgacaggacagcgaaaaaatacagaaacaaggggggaactgaaaagcttaggaatgcacagaacacccgcggggagacgaaaaaacc

SEQ ID NO: 168 - DNA, 314 bp, Pongamia pinnata
taggctaattcactttataacagtcaatgtgggctggggcttctagctcattttttcggctcctggtctctaccaaaaaaaaaaggtatcgtaagctagaggtcggggtcaggccctctaaaacgttttacccgcgcgactttgtcttcccctgcctatcctttcgtctaatccttccgacgcatcgaccgtatcccctctcttgccaatgccttgatttacttcctttattgctttactctacgacaactttccccattgtcccctgccatgaatctacttaaattctccaccaacccctagaacagttcggt

SEQ ID NO: 169 - DNA, 325 bp, Pongamia pinnata
caacgaaagacatggcataaagcgagctggagtcgagaatagagctgtactgactatttttattctcatttaaatttttgagagaaaaggaaaaaggagtcgtcgaacaatagatctctaacaactttagaaaagcataagcttgcaatgggctgaaaggacaataatgatcttattttgactgatattgacctgttccttgcaacaaattgataagcaatgctttcctataatgccagctttgtacaagaaagctgggtgggccctctagatctgcatgccttgcattctctaaagaaaaaatctttcatcaaagttgaaaatt

SEQ ID NO: 170 - DNA, 770 bp, Pongamia pinnata
aatagccgggaaaaaaacggctgttatacgatgccctcctcgctttgctcaatgaggttgttctactctcccaaaccaatcaatccttgttgagagaggttcccccccgcaataacgtttgtctccgtcactttgcattctccctcgtttaacaaactcctcaccggcggcgggtatcgcgcaagaataaaaatgtccggtattctccgcaatagtgtaacgtcaaactacactaacaaaatagttgaaagaagggttatcggacacttggctgattagcccatttgagtttgaagtatgatggtgtgaatcgtcatggattcgccttttttctcgcaaacagatcgaaacaaaaagaagtaactgagtttactacagcctggacttcagctttataaatgatgggctgtaggacagcgtcggttttaccaagtactgtaagaaaagaaacgtcacattaaaaaaagtaaagtatattttatagtgtttgataagagtgagaagtatggtccaaaagctgagaggtctccaagtaatgaaatacctgtaaagaattgaatggtggtggaaaaatgttgttgtgtaagtagataatgtcgtgtatcgagcgccgttggcgagtctgagggaggagatgtagcgtctttcagtgtagtgggagtcaaaggagcagaaggacagggtctctgtgccgctctgggacgtaccagaattccgcgcagcagagagaaagggccggcgggcgggtccatacttgcgtggaggacgag

SEQ ID NO: 171 - DNA, 135 bp, Pongamia pinnata
gggcggcttttttttcgttgcgtctcctcgccgggtcggggcttctggctctgaacactctgccaacccatgagctaggtttgtctccttgcagagctcctgctccactcgccaccctcctcccctctcctcttc

SEQ ID NO: 172 - DNA, 185 bp, Pongamia pinnata
gaaacgctaaattcgaatggtcagaggtcgcctggcctcggggttctatttttatgatttgggctggggtcagataaaaaagagaatatcgattttggggcttggggtcgggggtcccctctacttgccctttcctcacacccgcaccctctccatttcttccccacctacaatttacggttgac

SEQ ID NO: 173 - DNA, 184 bp, Pongamia pinnata
ggggggaaaggctttttggtagatttagacgcgctggtaccgaggtttttgttttaatgaataacgctgctggatgctaaaaagagaaaagagtctgaaggtagagtatcacacactaaagcaacagctagacccccccaacattgacgtggaggtggtcgattctcgggtacgtccttccgga

SEQ ID NO: 174 - DNA, 1023 bp, Pongamia pinnata
acgtaaagagaaaggggggggggagaaaaccggttttcaaataggcctccccagtgttcaagcagtcacagagggccactttcactatatcacggggactcctgttgtcagataggtctgagaattaatttgcgtagctttgcatcgctctcgttctaatgttaatgggtaaacactgtacatcgtggggtcgcgatgtccctgcgtacattgaattatgcgttcctcccagtaaggaaagctggctgcactttgatgggaccgcctgcaccttttgccggctgtggtgacagtgctttaatgctcagcagaacccccgcgacacccatgaaccgtgtaccaacctcgggaaacgtactcccggagagtgcctaccactattctgtctccggggagaaggtttttcgtccgtgaattcttaatcggctgcaatgaagcccgtaccagatttaccgctgtgatctagaattcttgaaatcaattattgcggtgggtacagtcagacgtgctggcgatttaattcggggacagtcgttgctgaggttgcactggcagcaccggttgtgttaatctgccaacactagttcgtagtcaatgaataagagcgctgataatttcggattgcgcactcccggcatgcgtcctcctatgctgtatctagagctagcgattcactctgaggctttctcgttgatttgtaggcctagtaagcttcgatctatcagttcaaatcgtcctctcgcgtgttcccttagtggttcggatgtgcttgcaccttgagagcatgtgcttagtttactttgaagttagagtcggcgtctcactctctctatcgagatcttgtctttccctctaaagtctcaacgtggagggaccggtcccacatggacaacagacacaatcagacagttcctttacgtcagcatggtctagtagcgattgacagtcagtgccggatcttaaactaatcggtagtcagtcatgtacatgtcacataccggtggtgagtcggacgataagatg

SEQ ID NO: 175 - DNA, 199 bp, Pongamia pinnata
gcgttaaccctggttcggtgactctgagtcgcagctactggggatttctctctgaggtccactgcagtcaaagaaaaaaaggcttttgttcgcatggccggttgagaacttaagaaaccgcccactccatttactgtttttaccgaactttcttttttcccccaatttttttttgttcttttatttttttttttttctt

SEQ ID NO: 176 - DNA, 203 bp, Pongamia pinnata
gggggggaataaaggttgattgaaccttgacgccgcaatctgggggcactcctctgatttactctcgcgattcgacaaaaaagaggatttctcgtggtaaacgagaggtatggaccactatctgcttatacaaatcccgtgttttcaacttttttactcccttccctgcctttgtactgtcccccgtattcttcattttttaa

SEQ ID NO: 177 - DNA, 1040 bp, Pongamia pinnata
aagggtacggttctcggatctctgcctagtctagattactctgtcattgtaaccttggtagtgtcctagtctgaaccttgttcttcctcgattccggccatggcttgcactctgaccaaggcgtcccctgaccctcatctgcccagcggagaacgacttaacactgatgctgaatcatttctaaagagagtgcgggttgtggacgggcatgtctccatgcatgtgctctcttccatgctatcacccgatccctctgctgacgctgctaattctctgcatgattcatccatgttctgtccctctttccctgtgtttgtttttcactattgaccatggggtctctttttgcgttgggatcgttcgaaagggcagattgtgtggaccggaaatggctgtctggcgagagacaggccatcaccccttgaatgcttttgttgaaaatgattgctgacttgccgatcccttcatcgatgctccggcgagacgtaaaccccgcttctgttaccgtttcattaagggtcacgtggtctctctacacgttgggatgtttcggaacggaagactgtgtgtacaggtaatgtatgtctggaaaaacagacaaaaccatcgcaaaagtaccattgcagcttataacgatcagtgggccgccttcgagagtgggcgaggtgtcaccgcacattgagttggtcggacaagccgacgatagtctctccaccaggactccacaacatgaagatacaggtgtacatgtatttgtttgtcatcccgctagcaaatctgcagatcaaagaaaatagataccgcctacgtggcttatctgtctgtcacaggccgcgtatgtggttaatgtgtttcactacatgctcataattctacttcgtgtcggtggcgcccacgatgttgtatcgaagcagtcaggatcatgcactgactcagagaatatctcgaaaaaagactatcgcgtacgggaagccttacgcgtcacgatgggtggacagcggggttacataggttaacggcattttcaagcataccgagtt

SEQ ID NO: 178 - DNA, 518 bp, Pongamia pinnata
ggatcctcccctttatcatccctcactccttctctaggcgccggaattagatctctcgaggttctagaccatggcatacccatacgacgtgcctgactacccctcccgtatccccgtagatgccggcacgagccgccgcttcacgccgccttccaccgccctgagcccaggcaagatgagcgaggcgttgccgctgggcgccccggacgcctgcgctgccctggccggcaagctgaggatcgtcgaccgcagcatggtggaggtgctggcgtgccaccggggcgagctggtgcgcaccgtcagccccatcttccggtgcgccgtgctgcgtatgcttggcgctggaattgaccgtggccagttctttcatgtgttggccctttgggaggttgtaaaggaccctctcatgacaggaacggatgacaatgatgaaaaatatgtggctgtgcccacaaatgggacgttatacatcaagaaacgtattagagataacgatgaaggtaggtttatctagcgtt

SEQ ID NO: 179 - DNA, 347 bp, Pongamia pinnata
aattagtttatttaagtgtctttcaccctcgtcaccggggtacccgtgccgggttactctcggtctgtagtagagaaaaaagcccctgcttaaaaagggcgatgacatctctaaaaggctattcctactacccaaacaagcataggtacccaaccgatgataaagtattttgtcagatgtcagtcccccataccagtaatcttagtacctcctaaatttcctgaggctccccctagaggtaaagacaaattacctttctccacacatcgccccccctgggcaatattacaggagagtggggggtatactgttcatcctgttcctgctcccttttctaccattcatca

SEQ ID NO: 180 - DNA, 234 bp, Pongamia pinnata
gggggagggcaataattaaatcttttattttatcgatggtgtgattccgattcttcattggtgatttattgtccctaatggcgacagggtcactgagatcgaacgaaatcaagttggactatgtgaccctactcctcactcgggtcgtcccgcatgacggactgctcacttacggttgggttgtccgacatcgccaacgtgccgtatgggaaagataggttccgccgattctgg

SEQ ID NO: 181 - DNA, 1022 bp, Pongamia pinnata
cggttgtcaattctgtgagtccgcggagtaccgggcgccgtcaggccctctattatttctcgagcttttggagtacgtcttctttacgttggggggaggggtatatgcgacagagtttcccctgctggaagagccccagacctttcctggacatttgtctggctgctcattctccttggaggtgcgctttttgagtttggatcttgggtctttttcgaccttccaaagagtgatgcagttttttaataccatttcagggggcttgaggcgcgccaccgacaacatgtgtaaaaaaaccggattaccccggtttttcctgggtgcgtaatgctgaaatatataaagccccgcgagttggccatacctgtgcacaggagaccactgtgttgcctgattgaccgtgcggggaaccgaaagcgagcgtagtctatcttgattgtgtttccgcgcactaaaaatgcacaaatttgcgttgttctatcttgacttacaggggtgaactaacatacctagagtggcaagggtccagtttgggctaggtctatggcacccggccgggatgaggacagaaaccagtcattgaagaagacgacatgatccatagtgtacatttatgtactgcagtccaaccgttgtagaacgctcttcagccccagtggaggcatccacaagaaacagactactgagagcattgcacatatgccatgcttgaaaatatatgaaccagctccctccctctgagcttcacctaaatgatctcggatgtgcttttgtgagatcgctacctcctctaacgagagcaataatggtcgacagctctgcatgctatattgaatgatagctcttgaagtatgtactattctggctgctcatgtcaccaagagacaaatgagtcaagtctaaccattctcttctctatgtactttgatggacgttctagccacaatccggtaggcatgtagtttactcagcataaatcctccctcgttcgcatcatgagactaagaaactgagaagaaaac

SEQ ID NO: 182 - DNA, 215 bp, Pongamia pinnata
agggatgaacactcgtttgtcgtctctgagcggcatgatgggggatttcttctgatagtcgcgctggccgtacactaaaaaaaaggggatggattcgtgcgggggtcggggaactttttttgatccccttgcaacatactactccgttgatttattgtctatatctattcaagtattaaataatgttgtcggtctccggttttccttattaaaaa

SEQ ID NO: 183 - DNA, 184 bp, Pongamia pinnata
agggtaaattcgtttttttgcactttccgccgcactaccgatggactcctttgataatactctcccagtaaaaaaaaaaagaggatttctccttgtacattagggggcgcgaccctccttgaagtccccgaccctccccacacacttgataacgtacgtcttctttcttgtcgcgttttcgtgg

SEQ ID NO: 184 - DNA, 224 bp, Pongamia pinnata
ggggttatgtttgtttgtgcccttttcagccctactcttgagggactttttgatggatagtctcctagagtaaaaaagagaggatttgtggtaactagggcgcgcccttttttaattttccccacttcccctcccttcccctcgtgacgtcttaaccccctaaatggtcccctgtccctgtactaaggccaaaccgtaatagtgccgtggacatgccttctagt

SEQ ID NO: 185 - DNA, 18 bp, Artificial sequence
  Primer
cagagagagagagagaga

SEQ ID NO: 186 - DNA, 18 bp, Artificial sequence
  Primer
gtgagagagagagagaga

SEQ ID NO: 187 - DNA, 18 bp, Artificial sequence
  Primer
aagagagagagagagaga

SEQ ID NO: 188 - DNA, 18 bp, Artificial sequence
  Primer
tcgagagagagagagaga

SEQ ID NO: 189 - DNA, 18 bp, Artificial sequence
  Primer
tagagagagagagagaga

SEQ ID NO: 190 - DNA, 18 bp, Artificial sequence
  Primer
aggagagagagagagaga

SEQ ID NO: 191 - DNA, 73 bp, Pongamia pinnata
gagggaagagagagagagagagaaagagaggtgtgggtgtgtggatgaaggaggggaagaagggtatttaggt

SEQ ID NO: 192 - DNA, 75 bp, Pongamia pinnata
taataataataatatgttttgttctaaaaagagagagagagagagacgatgacgtttattgtaaattataaattg

SEQ ID NO: 193 - DNA, 75 bp, Pongamia pinnata
ttgggaagttagagagagagagagagaggtaaaagaaaagaagtgagagacagagaaatataataaacgtacaag

SEQ ID NO: 194 - DNA, 73 bp, Pongamia pinnata; identical to SEQ ID NO: 191

SEQ ID NO: 195 - DNA, 75 bp, Pongamia pinnata
tatttaatgtataaaatttaaaatatatatatatatattatcttgaccggttcgaccctggttgaaccactaaac

SEQ ID NO: 196 - DNA, 75 bp, Pongamia pinnata
gttgcaaaaacctatttctctgtgttctgtttattgatatatatatatatatgtatctgccacctaaaccatgct

SEQ ID NO: 197 - DNA, 75 bp, Pongamia pinnata
cacaacctcaattgcattcaattaaacacacacacacacacaaacaaagcttattagttgacatacctttataga

SEQ ID NO: 198 - DNA, 75 bp, Pongamia pinnata
gcgaacgtacacacacacacacacacatagagaaatataaaaaaatctttttttaaaaaacgtttgagagtttgt

SEQ ID NO: 199 - DNA, 75 bp, Pongamia pinnata
ggctgcatcacctatcattctccaaaccctagctctctctctctctctcatctctctctcaaaaacttttacaag

SEQ ID NO: 200 - DNA, 75 bp, Pongamia pinnata
cctctcttctcacttgacacctctctctctctctcttggccaccattgattttccaacttcttttaagaagtttg

(19) United States
(12) Patent Application Publication
(10) Pub. No.: US 2014/0053294 A1
(43) Pub. Date: Feb. 20, 2014

(54) Pongamia Genetic Markers and Method of Use

(71) Applicant: Peter M. Gresshoff, Indooroopilly (AU)

(72) Inventor: Peter M. Gresshoff, Indooroopilly (AU)

(73) Assignee: The University of Queensland, Brisbane QLD (AU)

(21) Appl. No.: 13/885,886

(22) PCT Filed: Nov. 18, 2011
(86) PCT No.: PCT/AU2011/001496

§ 371 Date: Jul. 10, 2013

(30) Foreign Application Priority Data
Nov. 19, 2010 (AU) 2010905139

(51) Int. Cl. C12Q 1/68; C07H 21/04

(52) U.S. Cl. CPC C12Q 1/6895; C07H 21/04
USPC 800/265; 536/24.33; 536/23.6; 506/2; 435/6.12; 435/6.11; 800/260

(57) ABSTRACT

Primers suitable for nucleic acid sequence amplification of Pongamia plant genetic markers and a method of genetic analysis of Pongamia plants are provided. The primers comprise a repeat unit of two or three nucleotides repeated five to ten times together with a three prime extension of two or three nucleotides. Genetic markers amplified by the primers are also provided, from which may be produced further primers for genetic analysis of Pongamia plants. The primers, genetic markers and methods of genetic analysis may be suitable for selection and breeding of Pongamia plants having desired traits such as, or relating to, seed size, seed number, seed oil content, seed oil quality, seed flavour and toxicity, disease resistance, water use efficiency, nitrogen use efficiency, precocious flowering, flowering time, tree size, tree shape, growth rate, drought tolerance, salinity tolerance and/or growth in low-nutrient soils.

### TECHNICAL FIELD

THIS INVENTION relates to plant genotyping. More particularly, this invention relates to genetic analysis of Pongamia pinnata to identify genetic markers that correlate with one or more desired phenotypic traits.

### BACKGROUND

Pongamia pinnata (also known as Millettia pinnata) is a fast growing, deciduous tree that is an Indo-Malaysian species common in alluvial and coastal environments from India to Fiji including northern Australia, New Guinea, Malaysia, Southern China, Vietnam, and Indonesia. Pongamia pinnata is a “tree legume” in that it comprises Rhizobium-nodulated roots that enable symbiotic nitrogen fixation from sources such as atmospheric and soil-borne nitrogen. It also can use mineralised nitrogen in the form of nitrate.

Traditionally, Pongamia pinnata has been cultivated for ornamental gardens because of its attractive and abundant Wisteria-like flowers and abundant green foliage, and also for a variety of practical uses such as making cooking stove fuel, compost, strings and ropes and for extracting a black gum from its bark that is used to treat wounds caused by poisonous fish and in other traditional remedies. The seeds contain an oil (about 25-40% by weight) known as “pongam” or “honge” oil, which is a bitter, red brown, thick, non-drying, non-edible oil, which is used for tanning leather, in soap, as a liniment to treat scabies, herpes, and rheumatism and as an illuminating oil. This seed oil has a high content of triglycerides (containing up to about 55% oleic acid) which, in combination with the hardiness of the tree in poor soil conditions, has made Pongamia pinnata an attractive source of oil for the production of biofuels (e.g. biodiesel; Scott et al, 2008, Bioenergy Research 1 2-11).

With this in mind, there is a need to identify and select Pongamia pinnata plants that have genetically-linked traits associated with the optimal production of biofuels, such as high seed oil content. However, Pongamia pinnata is an outbreeding, genetically diverse species and there has been little previous study of this genetic diversity, particularly at the level of individual trees. A study described in Sahoo et al., 2010, Plant Syst. Evol. 285 121-125 used inter-sequence simple repeat (ISSR) analysis to examine genetic diversity between pooled samples from trees of different Indian regional sub-populations of Pongamia pinnata. The reported ISSR analysis utilised primers for nucleic acid sequence amplification that were arbitrarily designed to have nucleotide sequence repeats, with or without a single nucleotide 5′ extension, to enable randomly amplifying “inter-repeat” genomic sequences. These amplified genomic sequences were used to assess genetic diversity between the pooled Indian tree populations, although there was no attempt to correlate genotype with phenotype.

### SUMMARY

The present inventors have identified a need for more detailed genetic analysis of Pongamia pinnata, particularly with a view to understanding genetic variation underlying traits that are desirable for biofuel production, growth adaptation and overall plant performance. The previous study referred to above did not investigate genetic diversity between individual Pongamia pinnata trees and utilized sub-optimal primers for nucleic acid sequence amplification that were not refined to target repeat sequences that exist in the Pongamia pinnata genome. In principle, this invention is broadly adaptable to plants of other species of the Pongamia genus as well as Pongamia pinnata (also known as Millettia pinnata).

In a first aspect, the invention provides a method of producing an isolated nucleic acid suitable for nucleic acid sequence amplification, said method including the steps of: determining a genomic nucleotide sequence of a plant of the genus Pongamia according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4; and producing an isolated nucleic acid comprising said nucleotide sequence.

Suitably, the nucleotide sequence (Nₓ) is different to the nucleotide sequence (N)_{z}.

In a second aspect, the invention provides an isolated nucleic acid suitable for nucleic acid sequence amplification, said isolated nucleic acid comprising, or consisting of, a genomic nucleotide sequence of a plant of the genus Pongamia according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4.

Suitably, the nucleotide sequence (Nₓ) is different the nucleotide sequence (N)_{z}.

In one particular embodiment of the aforementioned aspects, x=2 or 3.

In another particular embodiment of the aforementioned aspects, y=8.

In yet another particular embodiment of the aforementioned aspects, z=2 or 3.

Specific embodiments of the isolated nucleic acid comprise a nucleotide sequence set forth in Tables 3, 4 and 5 (SEQ ID NOS:1-148).

In a third aspect, the invention provides a method of genetic analysis including the step of using the isolated nucleic acid produced according to the first aspect, or the isolated nucleic acid of the second aspect, to amplify a plurality of amplification products from a nucleic acid sample obtainable from a plant of the genus Pongamia.

In a fourth aspect, the invention provides a method of genetic analysis including the step of using one or more primers comprising respective nucleotide sequences of at least a portion of one of the amplification products obtainable by the method of the third aspect to amplify one or more further amplification products from a nucleic acid sample obtainable from a plant of the genus Pongamia.

In certain embodiments, the amplification products obtainable by the method of the third aspect comprise a nucleotide sequence set forth in any one of SEQ ID NOS:149-184.

In a fifth aspect, the invention provides an isolated nucleic acid comprising a nucleotide sequence set forth in any one of SEQ ID NOS:149-184, or a fragment or variant thereof.

In a sixth aspect, the invention provides a method of genetic analysis including the step of using one or more primers comprising respective nucleotide sequences of at least a portion of a nucleotide sequence set forth in any one of SEQ ID NOS:149-184 to amplify one or more further amplification products from a nucleic acid sample obtainable from a plant of the genus Pongamia.

In a seventh aspect, the invention provides a kit for genetic analysis of a Pongamia plant, said kit comprising one or more isolated nucleic acids (i) produced according to the first aspect; (ii) according to the second aspect or; (iii) of at least a portion of a nucleotide sequence set forth in any one of SEQ ID NOS:149-184 and one or more additional components suitable for genetic analysis.

In an eighth aspect, the invention provides a method of breeding a plant of the genus Pongamia, said method including the step of producing a progeny plant having a desired trait from one or more parent Pongamia plants, wherein at least one of the parent Pongamia plants is selected as having the desired trait by genetic analysis according to any of the aforementioned aspects.

Suitably, according to the aforementioned aspects the plant of the genus Pongamia is of the species Pongamia pinnata.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

Reference is made to the following Figures which assist in understanding non-limiting embodiments of the invention described in detail hereinafter wherein:

FIG. 1 shows selected SOLEXA 75 bp reads picked for PISSR primers design; Selected SOLEXA 75 bp reads picked for PISSR primers design. The sequences in targeted different repeats of nucleotide core units (GA, AT, CA, and CT), which are anchored either at the 3′ or 5′ termini of the repeats by a 2 to 3 nucleotide extension; Sequences are SEQ ID NOs:191-200 in order of listing.

FIG. 2 shows molecular diagnostics of PISSR markers using PAGE and silver staining. Left: original silver-stained polyacrylamide gel, M, molecular weight marker (bp); 1-9, individual Pongamia trees; Right: Partially enlarged polyacrylamide gel, clearly displaying polymorphic and conservative bands. The PCR products were amplified with primer PISSR4. The PISSR marker size ranges from 350 to 1,800 bp;

FIG. 3 shows molecular diagnostics of PISSR markers using capillary electrophoresis. This method is able to resolve fragments optimally in the size range of 80 to 400 bp by tagged fluorescent label HEX. Primer PISSR22 was used for displaying the genetic differences. The visualization of peaks is viewed in either a manner of semi-quantitative peak height or quantitative peak area. Position of red-coloured peak (ladder, from left to right): 350 bp; 360 bp. Position of green-coloured peak (from left to right): 346 bp, derived from both Pongamia trees G1-6 and G2-38 as conservative peak; 351 bp, Polymorphic peak in G1-6; 359 bp, Polymorphic peak in G2-38;

FIG. 4 shows genetic similarities of individual Pongamia trees from South-east Queensland and Malaysia based on PISSR markers;

FIG. 5 shows genetic similarity using the progeny derived from a single Pongamia mother tree (T1) based on multiple PISSR markers;

FIG. 6 shows reproducibility of PISSR markers derived from PISSR6 using clonal Pongamia trees. 1=mother tree W35; 2=clonal duplicate of W35; 3=mother tree W25; 4=clonal duplicate of W25;

FIG. 7 shows nucleotide sequences of “inter-repeat” genetic markers amplified by PISSR markers (SEQ ID NOS:149-184), including those referred to in Tables 6 and 7. Putative functional homologies to related sequences from M. trunculata, L. japonicus and/or Glycine max are also indicated.

### DETAILED DESCRIPTION

The present invention has arisen, at least in part, from the inventors' discovery of optimised nucleotide sequences comprising nucleotide repeat sequences with 3′ extensions useful in producing primers that facilitate nucleic acid sequence amplification-based genetic analysis of Pongamia pinnata plants. Surprisingly, the 3′ extension nucleotide sequence greatly enhances nucleic acid sequence amplification compared to the 5′ extension described in the prior art. The discovery of these optimised nucleotide sequences was assisted by deep sequencing of short fragments (˜75 bp) of the non-assembled genome of Pongamia pinnata to thereby produce primers that will specifically amplify target sequences present in the genome. Furthermore, primers comprising these optimised nucleotide sequences have proven useful in genetic analysis of Pongamia pinnata plants, resulting in the identification of multiple “inter-sequence” amplification products, at least some of which may be associated with desired traits in Pongamia pinnata plants. Accordingly, the invention enables genetic analysis and selection of Pongamia pinnata plants having one or more desired traits. The invention also provides a method of plant breeding that utilises these “inter-sequence” amplification products as genetic markers to assist in selecting parent plants for breeding progeny plants having a desired trait. Desired traits include seed size, number of seeds produced, seed oil content, seed oil quality, seed flavour and toxicity, precocious flowering, flowering time, tree size, tree shape, tree growth rate, disease resistance, drought tolerance, water use efficiency, nitrogen use efficiency, growth in low-nutrient soils, although without limitation thereto.

Therefore, in one aspect, the invention provides a method of producing an isolated nucleic acid suitable for nucleic acid sequence amplification, said method including the steps of: identifying a genomic nucleotide sequence of a plant of the genus Pongamia, preferably the species Pongamia pinnata, according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4; and producing an isolated nucleic acid comprising said nucleotide sequence.

In a related aspect, the invention provides an isolated nucleic acid suitable for nucleic acid sequence amplification, said isolated nucleic acid comprising, or consisting of, a nucleotide sequence of a genome of a plant of the genus Pongamia, preferably the species Pongamia pinnata according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form.

The term “nucleic acid” as used herein designates single- or double-stranded DNA or RNA and DNA:RNA and DNA:protein (PDNA) hybrids. DNA includes cDNA and genomic DNA. Genomic DNA includes nuclear, mitochondrial and chloroplast genomic DNA. RNA includes mRNA, cRNA, interfering RNA such as miRNA, siRNA, tasiRNA, and catalytic RNA such as ribozymes. A nucleic acid may be native or recombinant and may comprise one or more artificial or modified nucleotides, e.g., nucleotides not normally found in nature, for example, inosine, methylinosine, methyladenosine, thiouridine and methylcytosine.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences by hybridisation in Northern blotting, Southern blotting or microarray analysis, for example. Probes may further comprise a label, such as an enzyme (e.g. horseradish peroxidase or alkaline phosphatase), biotin, a fluorophore (e.g. FAM, ROX, TAMRA, Cy3, Cy5, Texas Red) or a radionuclide, typically to facilitate detection of the probe when bound to a “target” nucleic acid such as an amplification product.

A “primer” is usually a single-stranded oligonucleotide, preferably having 12-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. Typically, a primer comprises 15-30 contiguous nucleotides. The primer embodiments set forth in SEQ ID NOS:1-148 typically comprise 18-27 contiguous nucleotides. The primer may further comprise a label, such as described above, typically to facilitate detection of the primer.

As used herein, “hybridisation”, “hybridise” and “hybridising” refers to formation of a hybrid nucleic acid through base-pairing between complementary or at least partially complementary nucleotide sequences under defined conditions, as is well-known in the art. Normal base-pairing occurs through formation of hydrogen bonds between complementary A and T or U bases, and between G and C bases. It will also be appreciated that base-pairing, though weak and dependent on annealing conditions, may occur between various derivatives of purines (G and A) and pyrimidines (C, T and U). Purine derivatives include inosine, methylinosine and methyladenosines. Pyrimidine derivatives include sulfur-containing pyrimidines such as thiouridine and methylated pyrimidines such as methylcytosine. For a detailed discussion of the factors that generally affect nucleic acid hybridisation (such as salt, detergent, time, denaturant type and/or concentration, temperature, washing conditions etc.), the skilled addressee is directed to Chapter 2 of CURRENT PROTOCOLS IN MOLECULAR BIOLOGY Eds Ausubel et al. (John Wiley & Sons NY 1995-2009).

In particular embodiments, hybridization occurs under “stringent” conditions. Generally, stringency may be varied according to the concentration of one or more factors during hybridization and/or washing, such as referred to above.

Specific, non-limiting examples of stringent conditions include:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, washing is carried out at Tₘ=69.3+0.41 (G+C) %−12° C. In general, the Tₘ of a duplex DNA decreases by about 1° C. with every increase of 1% in the number of mismatched bases.

More specifically, the terms “anneal” and “annealing” are used in the context of primer hybridisation to a nucleic acid template for a subsequent primer extension reaction, such as occurs during nucleic acid sequence amplification or dideoxy nucleotide sequencing, for example. For a discussion of the factors that particularly affect annealing of primers to a complementary nucleic acid “template” during nucleic acid sequence amplification, the skilled addressee is directed to Chapters 2 and 15 of CURRENT PROTOCOLS IN MOLECULAR BIOLOGY supra.

By “nucleic acid sequence amplification” is meant a technique whereby a “template” nucleic acid, or a portion thereof, is used as the basis for a primer-dependent nucleotide polymerisation reaction that creates multiple nucleic acid “copies” of the “template” nucleic acid, or portion thereof. These techniques include but are not limited to polymerase chain reaction (PCR), ligase chain reaction, strand displacement amplification, rolling circle amplification, Q-β replicase amplification and helicase-dependent amplification.

An “amplification product” is a nucleic acid produced by nucleic acid sequence amplification. Amplification products may be detected or identified by any method known in the art, including staining, nucleotide sequencing and probe hybridization, although without limitation thereto.

In the context of an isolated nucleic acid comprising a nucleotide sequence according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4, the nucleotide sequence defined as 5′-(Nₓ)_{y}(N)_{z}-3′ comprises a repeat nucleotide sequence that comprises a repeat unit (Nₓ)_{y} wherein x=the number of same or different nucleotides in the repeated unit and wherein y=the number of times (Nₓ) is repeated in the nucleotide sequence. Suitably, (Nₓ)_{y} is a “tandem repeat” sequence without any intervening, non-repeated nucleotides. In an alternative less preferred embodiment, the repeat unit (Nₓ)_{y} is an imperfect repeat. For example, (Nₓ)_{y} may comprise one or more additional same or different nucleotides M that are not repeated, or are repeated to a value less than y.

Preferably, x=2 or 3 (i.e. a dinucleotide or trinucleotide repeat).

Preferably, y=7, 8 or 9.

It will also be appreciated that the nucleotide sequence defined as 5′-(Nₓ)_{y}(N)_{z}-3′ comprises a nucleotide sequence (N)_{z} located 3′ of the repeated nucleotide sequence, wherein z=the number of same or different nucleotides 3′ of the repeated nucleotide sequence.

Preferably, z=2 or 3.

Suitably, the nucleotide sequence (Nₓ) is different to the nucleotide sequence (N)_{z}.

In a preferred embodiment when z=2 or 3, (N)_{z} consists of N₁ and N₂ or N₁, N₂ and N₃, wherein N₂ is a different nucleotide than the second nucleotide of the repeat unit (Nₓ)_{y} to thereby prevent the inadvertent creation of an additional repeat within the 3′ extension. By way of example only, primer sequences conforming to this embodiment include (GA)₈GG (SEQ ID NO: 201) and (CA)₈CCT (SEQ ID NO: 21) whereas primer sequences not conforming to this embodiment include (GA)₈GA (SEQ ID NO: 202) and (CA)₈CAG (SEQ ID NO: 203).

Non-limiting embodiments of primer nucleotide sequences are set forth in SEQ ID NOS:1-148 (Tables 3-5).

Particularly preferred embodiments are provided in SEQ ID NOS:1-51.

Suitably, the isolated nucleic acid that comprises the nucleotide sequence according to 5′-(Nₓ)_{y}(N)_{z}-3′ as hereinbefore defined is a primer useful for nucleic acid sequence amplification, particularly for genetic analysis of Pongamia pinnata plants. Non-limiting embodiments of suitable primer nucleotide sequences are set forth in SEQ ID NOS:1-148 (Tables 3-5).

Accordingly a particular embodiment of the invention provides a method of genetic analysis including the step of using one or more of said primers to amplify a plurality of amplification products from a nucleic acid sample obtainable from a plant of the genus Pongamia, preferably of the species Pongamia pinnata. Nucleic acid samples may be obtained from any nucleic acid-containing part of a Pongamia plant inclusive of leaves, wood, seeds, flowers and roots, although without limitation thereto. Methods for obtaining nucleic acid samples are well-known in the art, although by way of example reference is made to Sahoo et al., 2010, supra, Murray & Thompson, 1980, Nucleic Acid Research 8 4321-4325 and Fulton et al., 1995, Plant Molecular Biology Reporter 13 207-209.

More specifically, the use of said one or more primers may facilitate nucleic acid sequence amplification of “inter-repeat” amplification products that may be used as genetic markers to assist in genotyping individual Pongamia pinnata plants, as will be described in more detail in the Examples. In typical cases, the method amplifies a plurality of “inter-repeat” amplification products that facilitate genetic analysis of individual Pongamia pinnata plants. By way of example only, a “fingerprint” typically comprising 10-30 amplification products may enable one individual plant to be distinguished from another, such as by identifying the presence or absence of one or more of the amplification products in one or the other plants.

It will also be appreciated that a nucleotide sequence of one or more “inter-repeat” amplification products may be determined, from which primers or probes may be designed and produced for nucleic acid sequence amplification or probe hybridisation, respectively.

Accordingly a further aspect of the invention provides a method of genetic analysis including the step of using one or more primers comprising respective nucleotide sequences of at least a portion of one of the amplification products obtainable by the method of the third aspect to amplify one or more further amplification products from a nucleic acid sample obtainable from a plant of the genus Pongamia.

In certain embodiments, the amplification products obtainable by the method of the third aspect comprise a nucleotide sequence set forth in any one of SEQ ID NOS:149-184.

Accordingly, another aspect of the invention provides an isolated nucleic acid comprising a nucleotide sequence set forth in any one of SEQ ID NOS:149-184, or a fragment or variant thereof.

By “fragment” is mean a single- or double-stranded portion or sub-sequence any one of SEQ ID NOS:149-184. Typically, a fragment comprises at least 10, 12, 15, 18, 20, 22, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400 or more contiguous nucleotides of any one of SEQ ID NOS:149-184. In one embodiment, a fragment is a primer suitable for nucleic acid sequence amplification.

By “variant” is meant an isolated nucleic acid comprising a nucleotide sequence at least 70%, 80%, 85%, 90%, 95%, 96%, 97%, 98% or 99% complementary to a nucleotide sequence of SEQ ID NOS:149-184, or a reverse complement thereof.

Another further of the invention provides a method of genetic analysis including the step of using one or more primers comprising respective nucleotide sequences of at least a portion of a nucleotide sequence set forth in any one of SEQ ID NOS:149-184 to amplify one or more further amplification products from a nucleic acid sample obtainable from a plant of the genus Pongamia.

The “inter-repeat” amplification products set forth in SEQ ID NOS: 149-184 are examples of amplification products obtainable by polyacrylamide gel electrophoresis (PAGE), DNA silver staining (Bassam & Gresshoff, 2007, Nature Protocols 2 2649-2654), excision from the PAGE gel and DNA sequencing, as described hereinafter in the Examples.

In a preferred embodiment, said one or more primers comprise respective nucleotide sequences of at least a portion of a nucleotide sequence set forth in any one of SEQ ID NOS:149-184. By this is meant that the primers comprise a nucleotide sequence of any one of SEQ ID NOS:149-184, or comprise a nucleotide sequence at least partly complementary thereto or at least partly complementary to a nucleotide sequence that is a reverse complement of any one of SEQ ID NOS:149-184. In this context, “at least partly complementary” means having sufficient complementarity to anneal or hybridize under stringency conditions that facilitate nucleic acid sequence amplification. Typically, base-pair mismatches may be tolerated, but primers would be at least 70%, 80%, 85%, 90%, 95%, 96%, 97%, 98% or 99% complementary to a “target” nucleotide sequence of SEQ ID NOS:58-92, or a reverse complement thereof.

Typically, the primers utilised according to these aspects (referred to herein as “inter-repeat primers”) are distinct from the primers defined by 5′-(Nₓ)_{y}(N)_{z}-3′, and are designed to specifically hybridise to nucleotide sequences in, or flanking, the corresponding genomic “inter-repeat” sequence. Such inter-repeat primers may be readily designed and created by persons skilled in the art. By way of example only, approaches to primer design are set forth in Chapters 2 and 15 of CURRENT PROTOCOLS IN MOLECULAR BIOLOGY supra.

In another particular embodiment, one or more probes that each comprise respective nucleotide sequences of at least one of the “inter-repeat” amplification products are used to hybridise to a corresponding nucleic acid in a nucleic acid sample obtainable from a plant of the species Pongamia pinnata. In this context a “corresponding” nucleic acid is a genomic DNA, cDNA or RNA that comprises a nucleotide sequence complementary to that of the probe. Typically, under hybridisation conditions of suitable stringency, the corresponding nucleic acid comprises a nucleotide sequence of, or complementary to, an “inter-repeat” sequence, as hereinbefore described. The corresponding nucleic acid would typically be a nucleic acid sequence amplification product.

A nucleic acid array may be particularly useful for “high-throughput” hybridisation analysis of nucleic acid samples obtained from Pongamia pinnata plants. Nucleic acid arrays are well-known in the art, although by way of example reference is made to Chapter 22 of CURRENT PROTOCOLS IN MOLECULAR BIOLOGY supra.

In this particular context, the invention provides a kit for genetic analysis of a Pongamia plant, preferably a Pongamia pinnata plant, said kit comprising one or more isolated nucleic acids, such as in the form of primers as hereinbefore described; and one or more additional components for genetic analysis. By way of example only, the one or more additional components may be for nucleic acid sequence amplification (e.g., a thermostable DNA polymerase) or other reagents such as restriction endonuclease(s), molecular weight markers and the like. The kit may further comprise detection reagents including one or more probes, DNA stains (inclusive of intercalating dyes), chromogenic or luminescent substrates or the like that facilitate detection of amplification products and/or probes hybridized to the amplification products.

A particularly advantageous embodiment of the invention provides “inter-repeat” amplification products that are genetic markers associated with, segregate with or are linked to, one or more desired traits of Pongamia pinnata plants. Non-limiting examples of desired traits include seed size, seed oil content (which varies from 25-40% by weight), seed oil quality (e.g., in terms of oleic, stearic and palmitic acid content), seed flavour and toxicity, precocious flowering, flowering time, tree size, tree shape, tree growth rate, disease resistance, drought tolerance, water use efficiency, nitrogen use efficiency, salinity tolerance, and growth in low-nutrient soils, although without limitation thereto.

The desired traits may be genetically “discontinuous” or “continuous”. In the case of a genetically “continuous” trait, an embodiment of the method of genetic analysis provides quantitative trait locus (QTL) analysis of Pongamia pinnata to thereby assess or determine the degree or extent to which each of one or more plant genetic elements (e.g., loci) contribute to the trait.

Accordingly, in a still further aspect, the invention provides a method of breeding a plant of the genus Pongamia, said method including the step of producing a progeny plant having a desired trait from one or more parent Pongamia plants, wherein at least one of the parent Pongamia plants is selected as having the desired trait by genetic analysis as hereinbefore described.

The one or more parent Pongamia plants may be different Pongamia plants or may be a self-fertilizing, individual parent plant.

By “breeding a plant”, “plant breeding” or “conventional plant breeding” is meant the creation of a new plant variety or cultivar by hybridisation of two donor plants, at least one of which carries a trait of interest, followed by screening and field selection. Generally, such methods include use of somatic or protoplast fusion, hybridization, reverse breeding, double haploids or any other methods known in the art. Typically, breeding methods are not reliant upon transformation with recombinant DNA in order to express a desired trait. However, it will be appreciated that in some embodiments, the donor plant may carry the trait of interest as a result of transformation with recombinant DNA which imparts the trait.

It will be appreciated by a person of skill in the art that a method of plant breeding typically comprises identifying at least one parent plant which comprises at least one genetic element associated with or linked to a desired trait. This may include initially determining the genetic variability in the genetic element between different plants to determine which alleles or polymorphisms would be selected for in the plant breeding method of the invention. This may also be facilitated by use of additional genetic markers (e.g., AFLPs, RFLPs, SSRs, etc.) associated with the desired trait that are useful in marker-assisted breeding methods.

By way of example only, a plant breeding method may include the following steps:
(a) identifying a first parent Pongamia pinnata plant and a second parent Pongamia pinnata plant, wherein at least one of the first and second parent plants comprise at least one genetic element associated with or linked to a desired trait;
(b) pollinating the first parent plant with pollen from the second parent plant, or pollinating the second parent plant with pollen from the first parent plant;
(c) culturing the plant pollinated in step (b) under conditions to produce progeny plants; and
(d) selecting progeny plants that possess the desired genetic element for a given trait.

It will be appreciated by those skilled in the art that once progeny plants have been obtained (e.g., F1 or BC (backcross) hybrids), which may be heterozygous or homozygous, these heterozygous or homozygous plants may be used in further plant breeding (e.g. backcrossing with plants of parental type or further inbreeding of F1 hybrids) or outbreeding.

One particular embodiment related to molecular marker development utilizes the progeny of an existing superior tree, treated as an F1 hybrid, and analyses co-segregation of molecular markers and one or more desired traits. Such association mapping is related to pseudo-testcrosses as for example described by Weeden (1994): pg 57-68. In: Plant Genome Analysis (CRC Press). Alternatively or in addition, even in the absence of the parent tree, the segregating population of seeds can be scored for association between molecular marker and desired trait.

Non-limiting examples of desired traits include seed size, seed oil content (which varies from 25-40% by weight), seed oil quality (e.g., in terms of oleic, stearic and palmitic acid content), number of seeds produced, precocious flowering, flowering time, tree size, tree shape, growth rate, drought tolerance, salinity tolerance, seed flavour and toxicity, disease resistance, water use efficiency, nitrogen use efficiency, growth in low-nutrient soils, although without limitation thereto.

So that preferred embodiments of the invention may be fully understood and put into practical effect, reference is made to the following non-limiting Examples.

### EXAMPLES

### Summary

Pongamia pinnata is a sustainable biofuel feedstock because of its abundant oil rich seed production, stress tolerance, and ability to undergo biological nitrogen fixation (minimizing nitrogen inputs). However, it needs extensive domestication through selection and genetic improvement. Owing to its outcrossing nature, Pongamia displays large phenotypic diversity, which is positive for selection of desirable phenotypes, and negative for plantation management. Variation was evaluated for mass, oil content and oil composition of seeds. To evaluate genetic diversity, and to lay a basis for a molecular breeding approach, we developed next generation sequencing (NGS)-derived ISSR markers (Pongamia Inter-Simple Sequence Repeats; PISSR). The special feature of PISSRs is that the number of nucleotide repeats and the 5′ and 3′ nucleotide extensions were not arbitrarily chosen, but were based on determined Pongamia genomic sequences obtained from a Pongamia NGS (Illumina®) database. Amplification products were separated by PAGE and visualized by silver staining, or by automated capillary electrophoresis to yield distinctive and reproducible profiles. Polymorphic bands were excised from polyacrylamide gels and sequenced to reveal similarity to DNA sequences from other legumes. We demonstrated: 1) a high abundance of nucleotide core repeats in the Pongamia genome, 2) large genetic and phenotypic diversity among randomly sampled Pongamia trees, 3) restricted diversity in progeny derived from a single mature tree; 4) stability of PISSR markers in Pongamia clones; and 5) genomic DNA sequences within PISSR markers. PISSRs provide a valuable biotechnology approach for genetic diversity, gene tagging and molecular breeding in Pongamia pinnata.

### Materials & Methods

### Plant Material and DNA Extraction

Plant material was collected from different locations in south-east Queensland (Australia) and the Kuala Lumpur region (Malaysia). To detect seed diversity, the seeds were germinated with 1:1 sand/soil in the glasshouse (18/6 h day/night cycle and 28° C./20° C. day/night temperature regime). Young leaf tissues visually clean and unaffected by pathogens were collected for DNA extraction from seedlings two months after germination.

Genomic DNA extraction was performed by a CTAB procedure (Murray et al., 1980; Doyle and Doyle 1987; Singh et al., 1999). The quality and quantity of the extracted DNA were confirmed by measurements with a ND-1000 Spectrophotometer (NanoDrop Products, USA).

### PISSR (Pongamia Inter-Simple Sequence Repeat) Primer Design and PCR

The approach utilized a Pongamia DNA sequence database recently generated via Illumina® Solexa GAIIx deep DNA sequencing technology at UQ. The database was based on a total genomic DNA library from a single Brisbane tree constructed from fragments of average 3 kb size, resulting in paired end reads each of 75 bp. The presence of dinucleotide repeats (e.g., CAₙ, GAₙ, ATₙ, CTₙ) in the Pongamia genome was determined by BLAST analysis (Altschul et al., 1990) of the database. From the paired end reads (75 bp), primers were designed on the basis of sequences containing eight repeats of dinucleotide core units with addition of the adjacent two or three nucleotides either at the 5′ or 3′ of the repeat (Table 5). PISSR primers were synthesized by Sigma-Aldrich®. PCRs were performed in a MJ Research thermal cycler with a thermal cycling profile consisting of denaturation for 3 min at 94° C., then 35 cycles of 45 s at 94° C., 30 s at the specific annealing temperature (this temperature varied depending on the % GC of the primer), and 1.5 min at 72° C., and a final extension cycle of 10 min at 72° C. Each PCR contained 1 unit of Taq DNA polymerase (Invitrogen, Carlsbad, USA), PCR buffer (20 mM Tris-HC1, pH8.4; 50 mM KCl), 0.2 mM dNTPs, 1.5 mM MgCl₂, 0.5 μM primers and 50 ng template DNA.

### PCR Product Detection by PAGE and Recovery of DNA Markers

PCR amplification products were separated by polyacrylamide gel electrophoresis (PAGE) using a Mini-Protean II cell (Bio-Rad, Hercules, USA) and visualized following silver staining (Bassam et al., 1991; Bassam and Gresshoff, 2007). Separation was in 0.45 mm thick, 7.5×10 cm vertical slab gels of 5% polyacrylamide backed on GelBond PAG polyester film (Lonza, Rockland, USA) in TBE buffer, until the dye front reached the end of the gel. The polyester-backed polyacrylamide gels were air-dried and stored in a photo album as a “molecular archive”, whereupon DNA fragments of interest were extracted, re-amplified, cloned and sequenced.

Small pieces of polyacrylamide gel containing the desired DNA fragment were carefully excised from dry or fresh gels with a sterile scalpel. In the case of dry gels each gel piece was cleaned by soaking in 95% ethanol. A scalpel was used to sharply delimit the desired DNA fragment, and the excised gel piece was then rehydrated (if needed) in 10 μl of sterile water. The gel segment was next placed in 20 μl of PCR reagents with the same primer as was used to generate the relevant DNA marker. Re-amplified PCR products were separated by PAGE and visualized by silver staining, as previously. Purified PCR products were then further characterized by DNA sequence analysis.

In addition, capillary electrophoresis (CE) was done by a MegaBACE™ 1000 capillary system (GE Healthcare Life Science, Piscataway, USA).

### Analysis of Genetic Similarity

PISSR polymorphic markers were scored manually using a binomial ‘1’ and ‘0’ matrix for their presence and absence, respectively. The level of genetic similarity among Pongamia individuals was established by clustering method UPGMA (unweighted pair-group arithmetic average) with the SHAN subroutine, through the software NTSYS-pc version 2.0. Dendrograms were used to represent the genetic relationship among the 29 local Pongamia trees.

### Analysis of Seed Oil Content and Composition

Seed oil was extracted by the chloroform/methanol extraction procedure (Schmid 1973; Christie 1993) using finely chopped individual seeds. Fatty acids were analysed using gas chromatography (Shimadzu GC-17A, Japan) on a DB-23 60 m×0.25 mm×0.25 μm capillary column with GC-FID (Shimadzu Co., Japan) by Analytical Services, School of Agriculture and Food Science, UQ.

Bioinformatics Analysis with DNA Sequence of the PISSR Markers

DNA sequencing was performed at the Australian Genome Research Facility (AGRF), The University of Queensland. Bioinformatics analysis of the DNA sequences from PISSR markers was performed using public databases such as NCBI (www.ncbi.nlm.nih.gov); Gene Indices (compbio.dfci.harvard.edu/tgi/cgi-bin/tgi/Blast/index.cgi); Lotus japonicus EST index (est.kazusajp/en/plan) or Phytozome (www.phytozome.net/soybean). From these databases, a BLAST-search of DNA sequences amplified by PISSR markers identified putative Pongamia genes. The DFCI gene indices database provided access to the UniProtKB/Swiss-Prot database (www.uniprot.org) to allow for more insight of functional similarities if markers were related with protein-encoding sequences.

### Results

### Phenotypic Diversity of Pongamia

The genetic diversity in a randomly chosen set of Pongamia trees was reflected in distinct phenotypic differences at the gross level, including whole tree architecture and leaf morphology between south-east Queensland street trees T10 and GC2, for example. Furthermore, seed-derived Pongamia trees, planted for a life cycle analysis of Pongamia at the UQ Gatton campus, showed diversity for flowering time with 6% of trees flowering and setting seed precociously by 15 months of age. Significant differences in seed size, shape and weight were also observed (Tables 1 and 2). Seed oil analysis showed variation of oil content and composition between trees and between progeny seeds of a single parent tree, T10 (Tables 1 and 2). For individual seeds from 10 randomly selected Pongamia trees, the seed mass, oil content and oleic acid/oil content varied from 0.41-1.5 g, 19.7-50.5% and 25.4-54.2%, respectively. The lower values appear to be derived from a set of distinct Pongamia trees (OT1, GC1, GC2, GC3; see Table 1), possibly belonging to an as yet defined sub-species. In contrast, six progeny seeds of tree T10 (a high performer) showed less variation for seed mass, oil content and oleic acid/oil content (0.97-1.37 g/seed, 40.3-52.3% per seed and 51.6-68.3% oleic acid content). The results from seed oil analysis suggested that the variations of seed oil content are larger between seeds from different trees than between seeds from the same parent tree (Tables 1 and 2).

### PISSR Primers Generated Extensive Polymorphic Bands

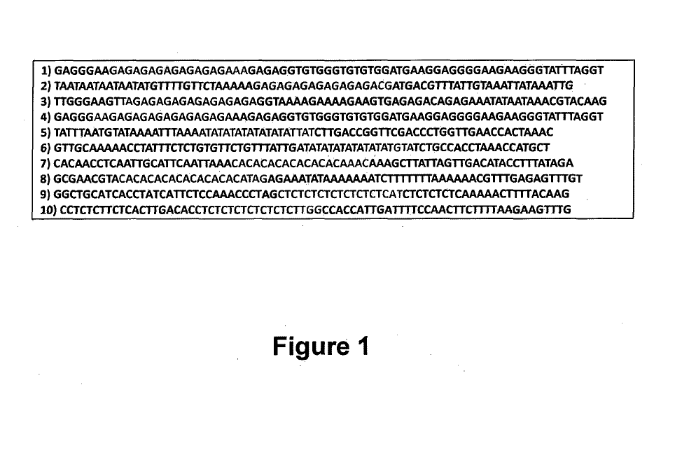

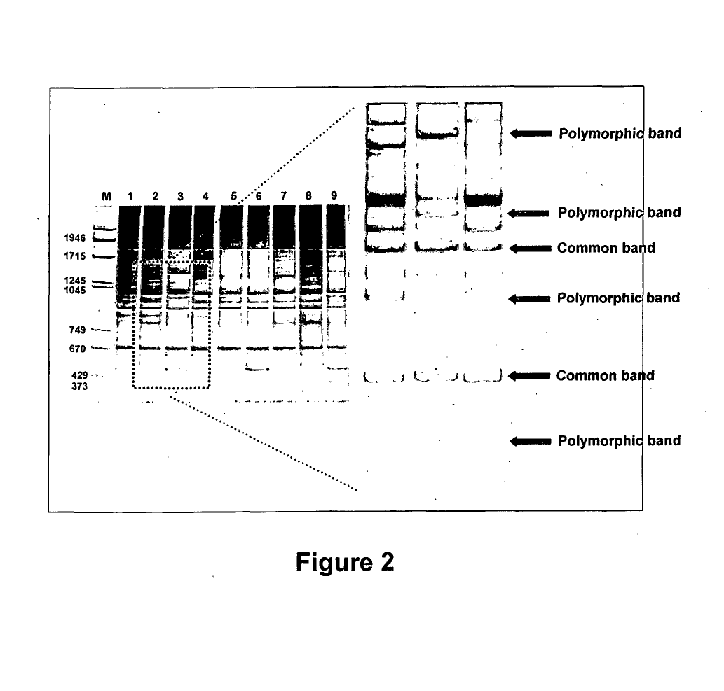

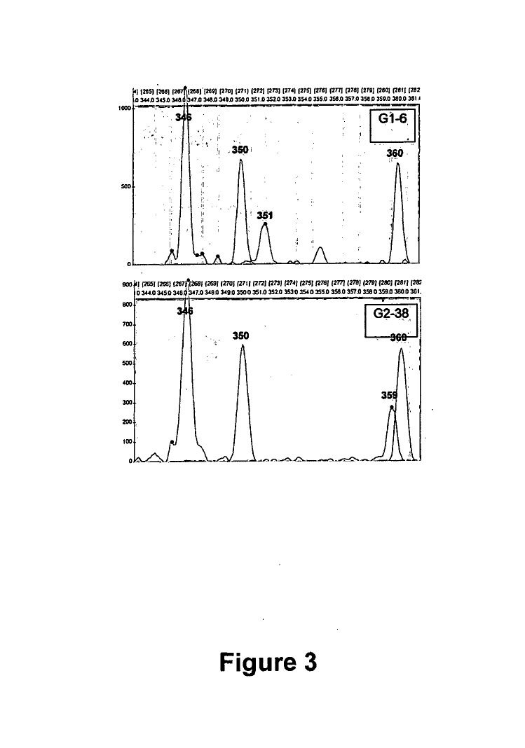

A total of 27 PISSR primers were tested in this study, as listed in Tables 3-5. All tested primers were based on a (GA)₈ (SEQ ID NO: 204) or (CA)₈ (SEQ ID NO: 205) motif, with an additional 5′ or 3′ di- or tri-nucleotide extension. These extensions were based on flanking DNA sequences from the paired-end Illumina® reads to limit the number of amplicons for diversity scoring and assessment. Although not utilized in this study, many 1 to 3 nucleotide core unit tandem repeats were identified in the NGS database. FIG. 1 displays the DNA sequence of a selection of 75 bp reads and illustrates the typical di-nucleotide repeat sequences found in the Pongamia genome. Of the 27 primers tested, 23 had a 3′ extension and four had a 5′ extension. Importantly, all primers successfully enabled the reliable amplification of numerous DNA fragments (e.g., up to 23 bands for primer PISSR1; Table 4). Interestingly, all ISSR primers tested that had a 5′ extension, were able to generate only common bands with no polymorphic markers able to discriminate between accessions, whereas all PISSR primers with a 3′ extension were able to generate polymorphic markers. As an example, FIG. 2 demonstrates the typical profile of common and polymorphic amplicons, in this case derived from nine local Pongamia trees using the primer PISSR4 ((GA)₈TG; SEQ ID NO: 4). Resolution of amplicons by PAGE and silver staining enabled the routine scoring of bands in the size range of 250 to 1,900 bp.

To test the robust application of the methodology described above for the assessment of Pongamia genetic diversity, DNA was extracted from 29 trees, 26 from south-east Queensland and three from Malaysia. Genetic relatedness was determined following PCR with 12 PISSR primers (Table 4). The DNA profiles obtained by PAGE and silver staining were highly reproducible with clearly definable bands being scored as either conserved or polymorphic markers. Of the 12 PISSR primers used in this part of the study, 10 primers produced 105 conserved and polymorphic DNA fragments with apparent sizes from 250 bp to 1.9 kb. The number of reproducibly visible bands ranged from 10 to 23 for each primer (Table 4). From the pool of conserved and polymorphic amplification products, 7 to 15 polymorphic markers were generated per PISSR primer. The highest level of polymorphism (i.e., 75%) was detected with the primers PISSR1 (GA)₈AT (SEQ ID NO: 1) and PISSR18 (CA)₈ATT (SEQ ID NO: 12). The number and size of the amplicons suggested that the PISSR primers were able to generate markers with a wide distribution and location in the genome of Pongamia.

CE was able to resolve fragments optimally in the size range of 80 to 400 bp. Detailed resolution of PISSR markers using CE was demonstrated (FIG. 3). Table 5 lists the number of common and polymorphic DNA markers amplified from genomic DNA of 22 selected field samples with 8 PISSR primers. Due to the higher resolving power of CE, the laser detection enabled identification of markers with a minimal difference of 1-2 bp (FIG. 3). Thus CE offered maximal resolving power with more polymorphisms compared to PAGE/SS, but over a smaller size range. As an example, 53 polymorphic markers were generated from 22 Pongamia samples with primer PISSR22 bp CE (Table 5), being almost three times more than those generated from PAGE/SS, even though the effective size detection range was restricted in CE. With primers PISSR 14, 17 and 18, the marker size ranged from 80-400 bp and 400-1,900 bp for CE and PAGE/SS, respectively. These results suggest that a combination of both approaches is able to obtain more extensive polymorphic markers.

### Genetic Similarity Analysis

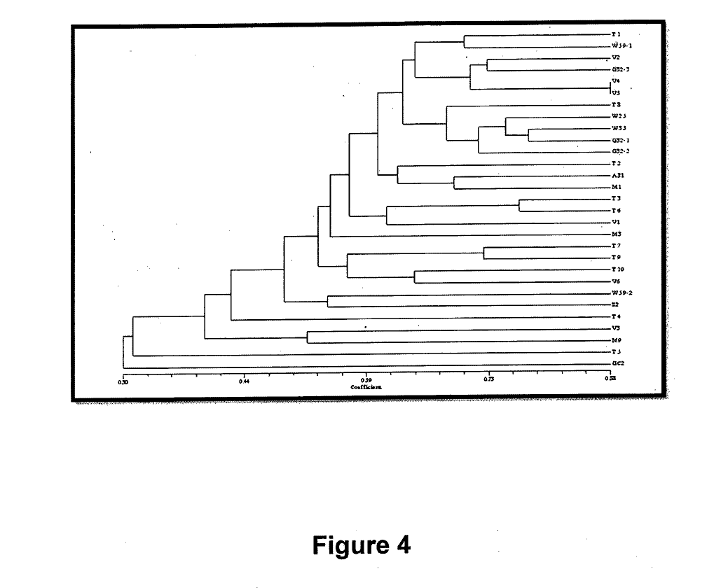

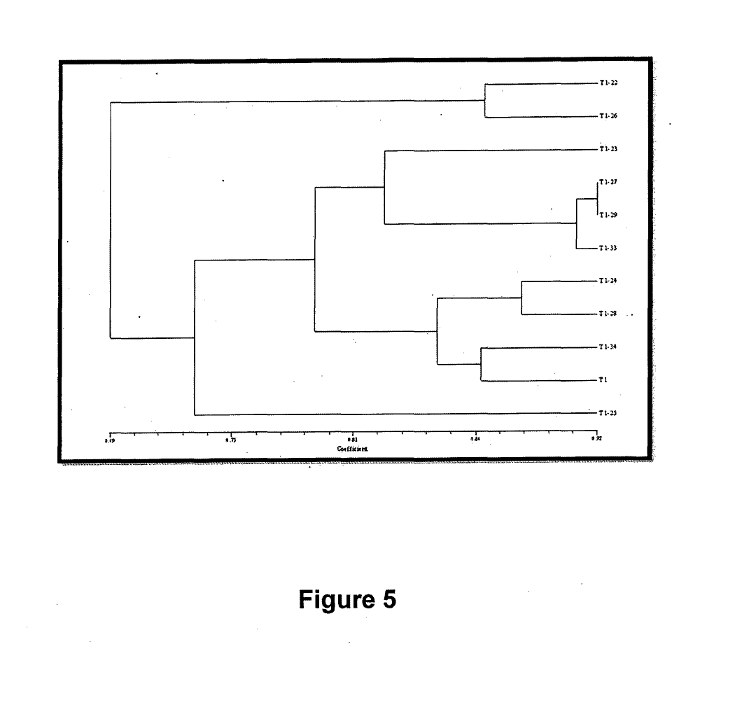

Binomial scoring for the presence (1) or absence (0) of the 105 polymorphic markers generated a quantitative assessment of genetic similarity/diversity for 29 randomly selected trees. The Jaccard's similarity coefficient ranged from 0.30 to 0.88 (FIG. 4). UPGMA cluster analysis indicated that there was no correlation between the location of Pongamia trees and genetic similarity (FIG. 4). For example, three Malaysian trees (M1, M3 and M9) were genetically interspersed amongst the remaining South-east Queensland trees. Malaysian tree M1 and Queensland tree A31 were in a cluster with a coefficient value of 0.67, while trees M9 and V3 were in another cluster with a coefficient value of 0.51. The reproductive origin of these trees is not known, but it is likely that each tree was grown from a seed, in part an explanation for their wide genetic diversity. Despite the relatively wide diversity amongst the tested accessions, this analysis generated a ‘single rooted phylogenetic tree’ (FIG. 4), suggesting a common origin for Pongamia.

The PISSR approach demonstrated the outcrossing nature of Pongamia and the subsequent genetic variation between a parent plant and its seed-derived progeny. Four PISSR primers were used to characterize a single mature tree (T1) and ten progeny saplings; forty-six polymorphic markers were generated. The similarity coefficients for the parent tree and its progeny ranged from about 0.69 to 0.92 (FIG. 5). More specifically, sapling T1-34 was most closely related to the parent tree T1 (similarity coefficient value 0.86). Saplings T1-24 and T1-28 were similarly highly related (0.88), while T1-27, T1-28 and 11-33 showed the highest value of greater than 0.92). T1-25 was the most distantly related sapling (0.73). Despite this level of genetic variation the parent tree T1 and its progeny were overall more closely related than tree T1 was to the other 28 Pongamia accessions described above (FIG. 4).

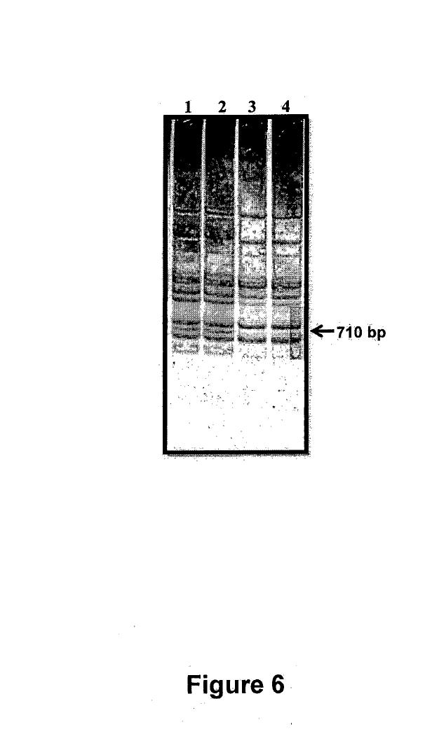

Vegetative propagation through grafting, cuttings and/or tissue culture is an effective way to expand the numbers of elite Pongamia lines for large-scale, broad acre plantings (Biswas et al., 2011). To confirm the reliability of the PISSR marker approach, two clonal trees from rooted cuttings of parent trees W35 and W25 were tested. W25 and W35 DNA differed in one distinct polymorphic marker at 710 bp. As expected, this polymorphic marker together with other conserved DNA fragments were maintained in clonal replicates (FIG. 6).

### Annotation and Association of PISSR Markers

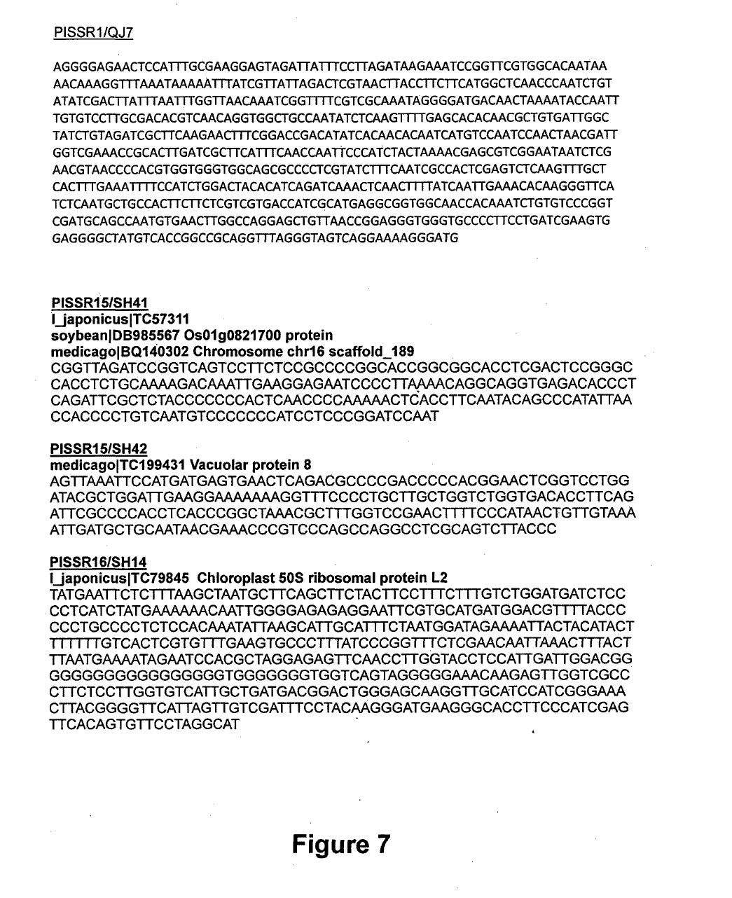

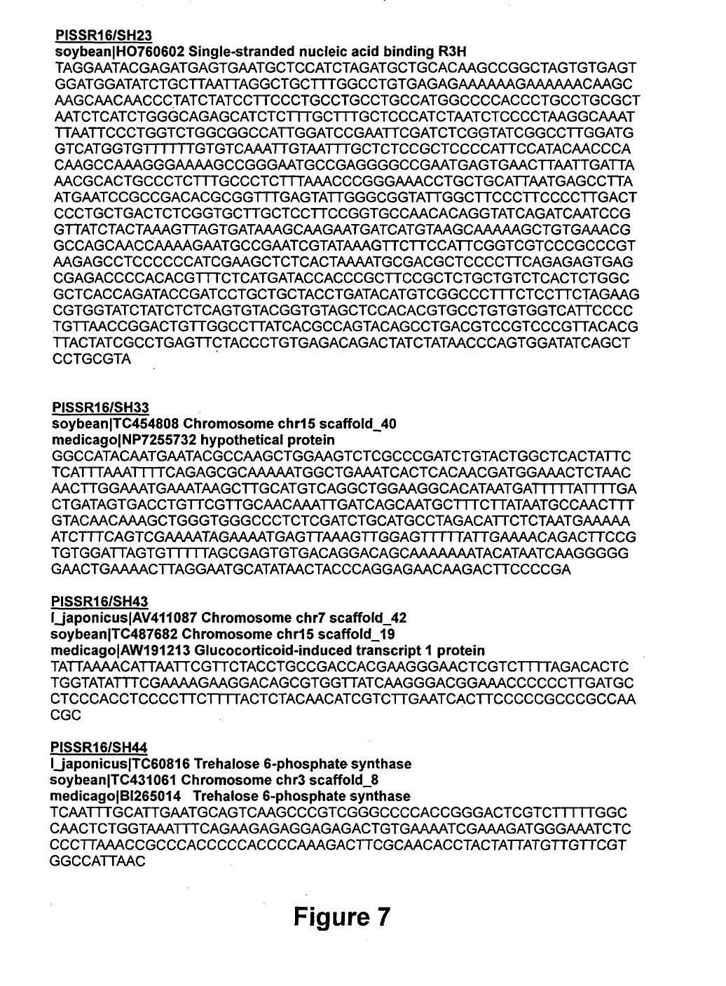

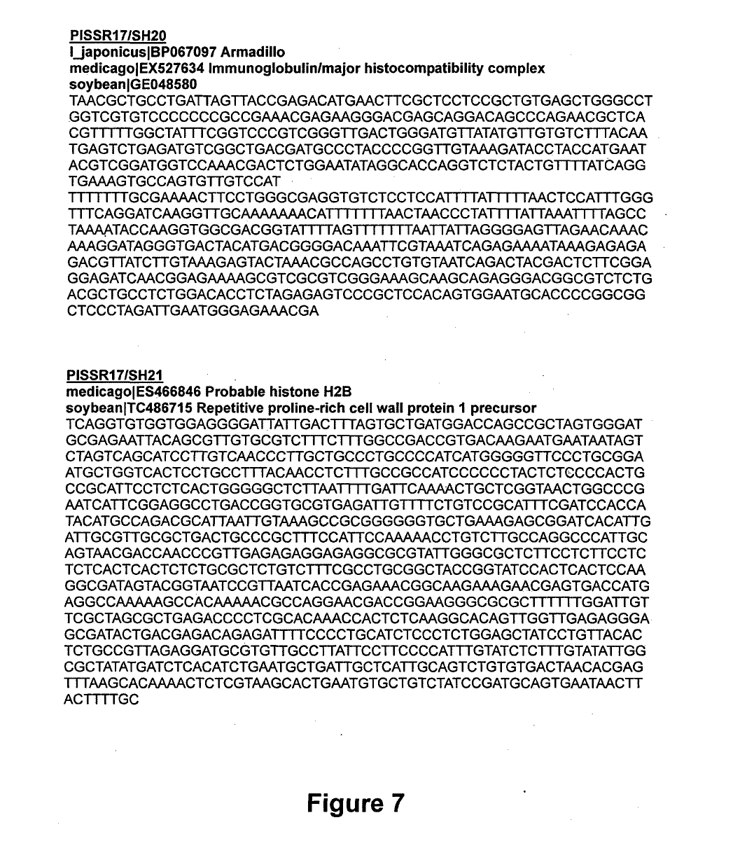

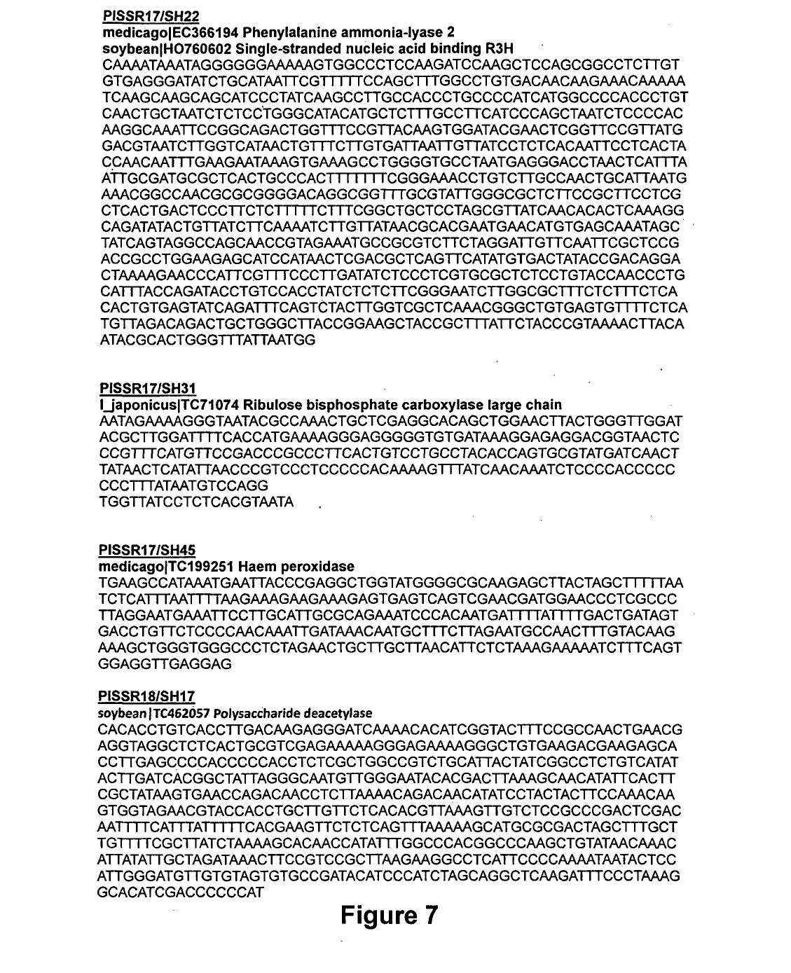

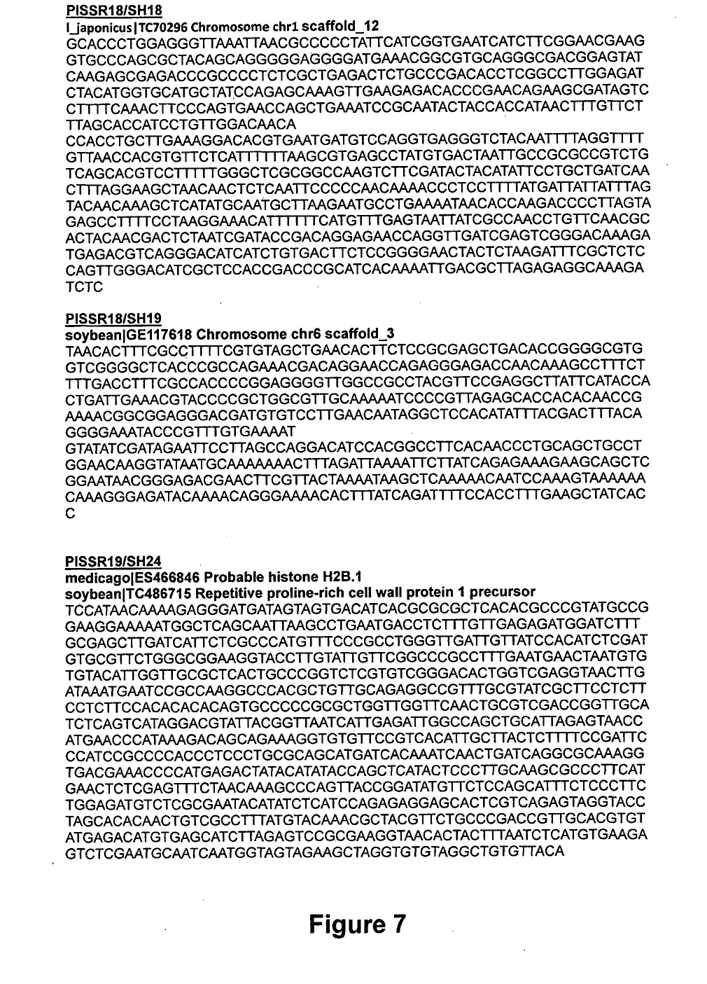

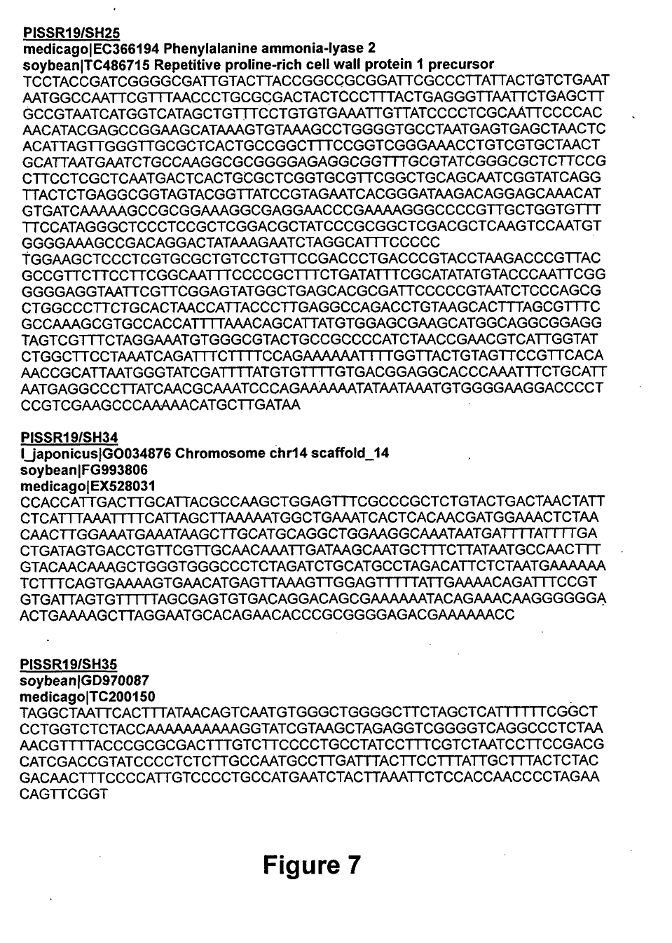

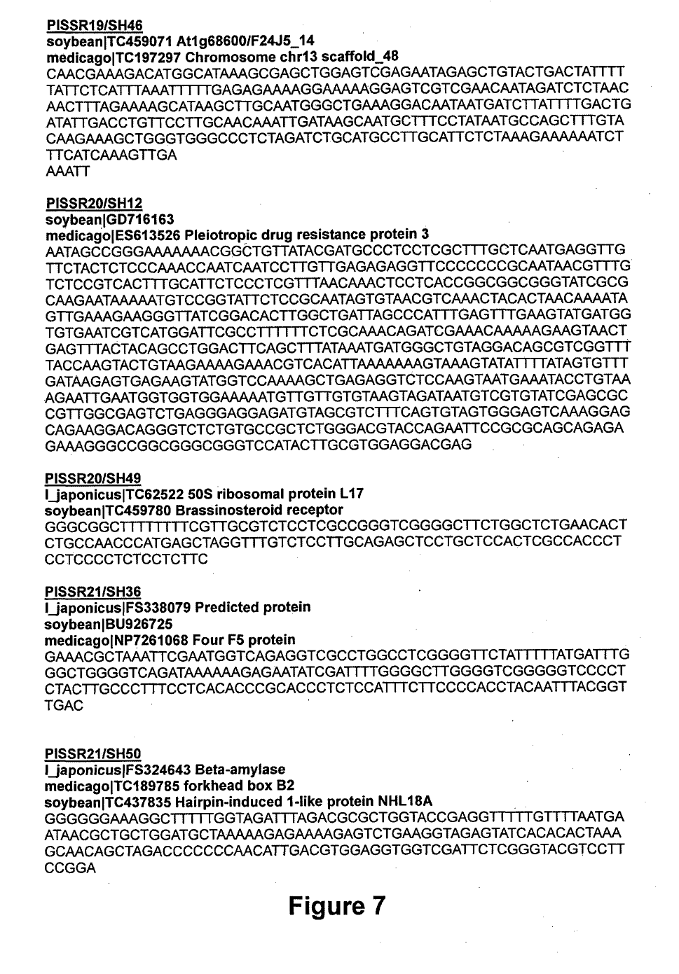

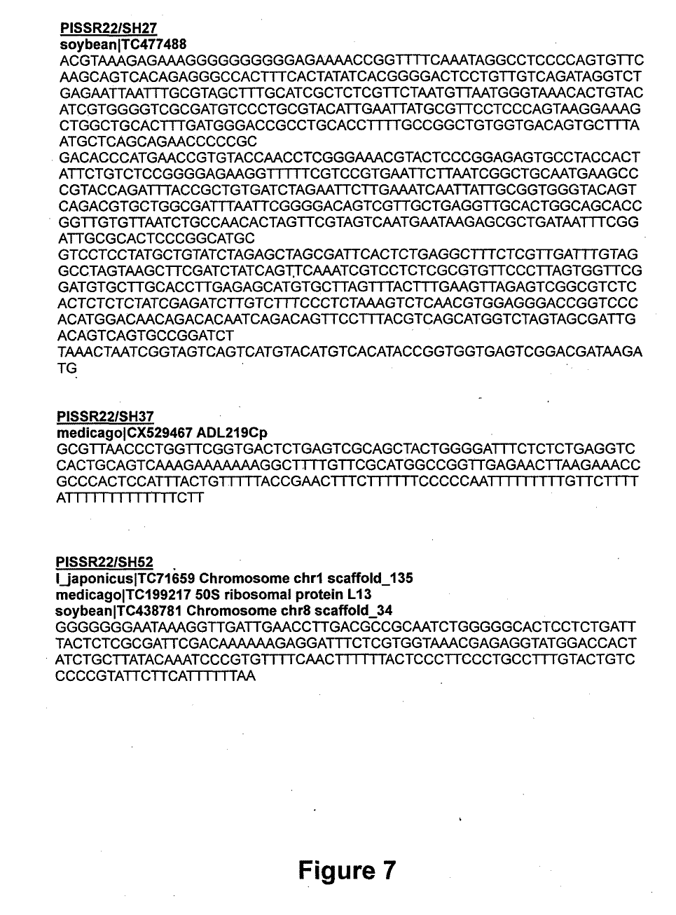

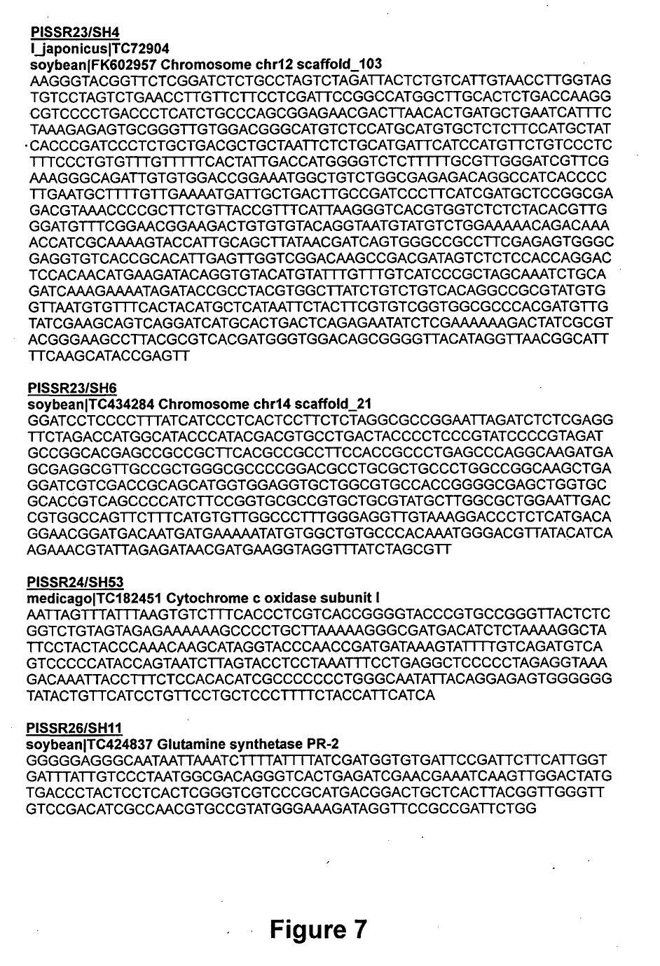

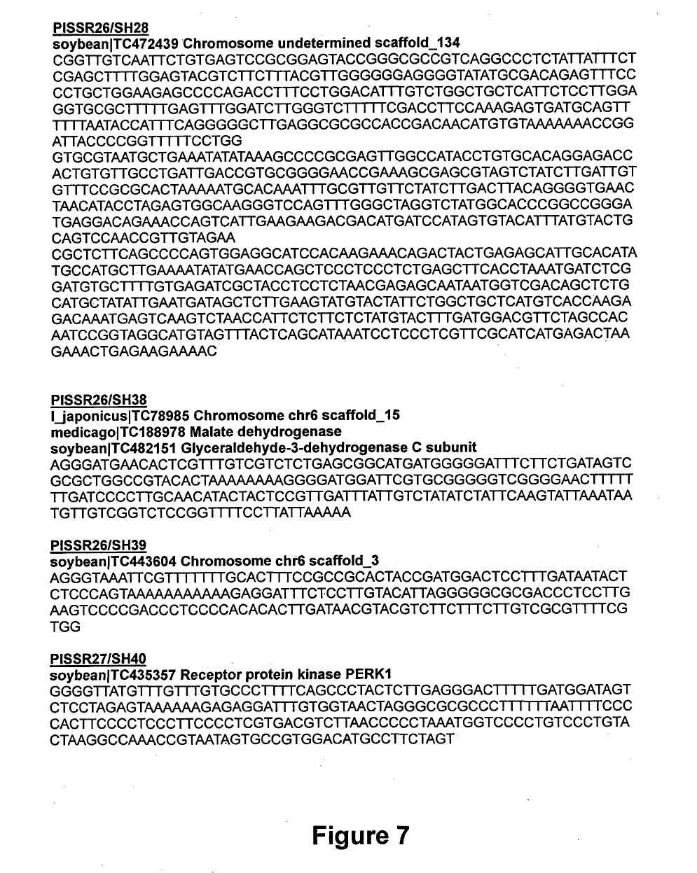

DNA fragments generated by PISSR primers were physically recovered from silver stained polyacrylamide gels and purified for DNA sequencing. Comparative analysis of the derived DNA sequences was performed relative to sequences deposited in the public databases NCBI (www.ncbi.nlm.nih.gov/), UniProtKB/Swiss-Prot (www.uniprot.org/), Phytozome (www.phytozome.net/soybean), Lotus EST index (est.kazusa.or.jp/en/plant/lotus/EST/index.html), or the Gene Index Project (compbio.dfci.harvard.edu/tgi/tgipage.html). Table 6 shows the nucleotide sequence relatedness of PISSR markers to genomic sequences of L. japonicus, G. max (soybean) and M. truncatula. These data were selected and tabulated from the greatest high-scoring segment pairs (HSP) searched within the genome of each species. These similar sequences came mainly from soybean, secondly in M. truncatula, and least from L. japonicus genomes (reflecting the levels of complete genome sequence determination of these legumes). This result suggested that it is possible to BLAST-search public DNA sequences from PISSR markers at the levels of DNA, cDNA and amino acid sequence to search for potential gene similarities in Pongamia. Those marker sequences (shown in Table 6) were further analysed to infer their possible functional annotation related to sequences in L. japonicus, soybean, and M. truncatula (Table 7). All Pongamia sequences referred to in Tables 6 and 7 are set forth herein in FIG. 7 (SEQ ID NOS:149 to 184). Each of these sequences may be used for the construction of primers whereby amplification of PISSR markers, or fragments thereof, may be used to identify the presence of these markers in Pongamia pinnata plants.

### Discussion

Phenotypic diversity was easily determined in Pongamia pinnata plants. Here we demonstrated quantitatively the degree of variation in terms of seed size, seed oil content and seed oil composition (as indicated by oleic acid, C18:1). In parallel, molecular marker technologies were developed to give clear and more direct information of the genetic polymorphisms distinguishing particular accessions of Pongamia.

Advantages of many molecular marker techniques are that (i) no prior genomic sequence information is required, (ii) markers are stable, (iii) they are detectable at all developmental stages of an organism, and (iv) they are not cell specific (Agarwal et al., 2008). We advanced on these positive attributes of molecular marker technology as the Australian Centre for Plant Functional Genomics (ACPFG) and the ARC Centre of Excellence for Integrative Legume Research (CILR) created a Pongamia DNA SOLEXA-GAII database. The database provided 2.9×10⁷ (29,474,558) Pongamia short reads which was used to design PISSR primers. Thus, the special feature of PISSR primers is that the number of repeats of nucleotide core units and anchored 5′ and 3′ nucleotide residues of PISSR primers represented real Pongamia genome sequence. Therefore, PISSR primers are significantly distinguished from arbitrary ISSR primers, as reported by Zietkiewicz et al. (1994). We conducted a BLAST search of our Pongamia GAII database for different nucleotide core units (GA; CA; TA: AT) with different numbers of repeats and then designed PISSR primers according to needs.

The separation of complex DNA samples with high resolution by polyacrylamide gel electrophoresis (PAGE) has broad application. DNA silver staining has proven a very effective visualization method offering superior clarity and sensitivity (Bassam and Gresshoff, 2007). Amplifications with ISSR primers were usually resolved by agarose gel electrophoresis and ethidium bromide (EB) staining (Wolfe et al., 1998; Sahoo et al., 2010) or resolved by PAGE and visualized by autoradiography (Zietkiewicz et al., 1994). However, Gonzalez et al. (2005) used large acrylamide gels (380×320 mm) and silver staining to separate and visualize ISSR amplification products, allowing the distinction of sympatric wild and domesticated populations of common bean. Here we used both ‘mini-PAGE’ (100 mm×80 mm) and silver staining methods to separate the PCR products amplified by PISSR primers. The advantages of PAGE/SS over agarose gels and ethidium bromide staining were obvious, as PAGE/SS displayed clear and sharp images, and highly sensitive visualization on polyacrylamide gels (FIG. 2). Thus PAGE/SS was selected as a part of PISSR marker detection, as it allowed robust PISSR detection as well as subsequent band sequence determination.

Zietkiewicz et al. (1994) stated that the 3′ anchored arbitrary ISSR primers of (CA)₈RG or (CA)₈RY, in which R stands for either purine and Y for either pyrimidine, resulted in marker sizes from 200 to 2,000 bp in various eukaryotic species. Table 4 showed that PISSR primers produced numerous markers with a similar size range. For example, primers (GA)₈AT (SEQ ID NO: 1) and (GA)₈AA (SEQ ID NO: 2) produced PISSR markers ranging from 250 to 1,900 bp. To expand the PISSR primer range (with a sequence of (GA)₈ (SEQ ID NO: 204) and two nucleotide extensions; ((GA)₈+2), primers carrying a (CA)₈ (SEQ ID NO: 205) core unit and a three nucleotide extension at their 3′ termini [‘(CA)₈+3’] were generated and produced abundant markers. For ‘(CA)₈+3’ primers the smallest reliably detected fragments were 400 bp (instead of the 250 bp seen for (GA)₈+2) and the percentage of polymorphic markers was fractionally lower than the average for the former set of primers. This suggests that there is a more stringent (locus specific) PCR amplification when using PISSR primers with longer nucleotide extensions at the 3′ terminus.

The phylogenetic tree diagrams (FIGS. 4 and 5) were made on the basis of the presence or absence of the markers identified in ISSR amplicons. The dendrogram exhibited at least nine clusters with different coefficient values from 0.3 to 0.88, suggesting large genetic variation of the individual Pongamia trees from South-east Queensland, Australia and Kuala Lumpur, Malaysia, based on 105 PISSR markers (FIG. 4). As three Malaysian samples were classified to three clusters with some Queensland Pongamia trees, there is no evidence, at least from this study, indicating a correlation between geographic location and genetic similarity. However, we make this conclusion in the knowledge that we cannot describe in detail the ancestry of these tested Pongamia trees.

As described, the Jaccard's similarity coefficient ranged from 0.30 to 0.88 among the 29 Pongamia trees (FIG. 4). In contrast, coefficient values for DNA products from progeny saplings T1 ranged from just 0.69 to 0.91 (FIG. 5). The coefficient value range in T1 seeds demonstrated the closer kinship between T1 seeds and its parent than the relatedness between randomly selected trees (FIG. 4). We conclude that PISSR polymorphisms occurred frequently among Pongamia individuals, but less so between related progeny, factually supporting presumed outcrossing breeding in Pongamia.

Previously reported ISSR analyses utilized DNA amplification primers that were arbitrarily designed to have nucleotide sequence repeats, with 1-3 nucleotides on the 3′ or 5′ termini, to enable randomly amplifying “inter-repeat” genomic sequences. A study described by Sahoo et al. (2010) used inter-sequence simple repeat (ISSR) analysis to examine genetic diversity between pooled samples from trees of different geographic locations in India. These amplified genomic sequences were used to assess genetic diversity between pooled Indian tree populations, but there was no attempt to correlate genotype with phenotype. However, our analysis correlated the extreme (outlier) genotype of Queensland Pongamia tree GC2 with its unique phenotypic characteristics (oil content and composition, leaf shape, seed shape, growth habit). This result means that PISSR amplification profiles from GC2 reveal polymorphic markers in concert with its phenotypic traits.

The PAGE and CE are specialized in separation of DNA products with different size range, in this study 250 to 1,900 bp and 80 to 400 bp, respectively. CE offered higher resolving power than that of PAGE, but the range of marker size was more limited. Throughout the analysis of PAGE and CE, DNA markers generated by the majority of PISSR primers generated a reasonably even distribution across the range of sizes for both PAGE and CE (Tables 4 and 5). Hence both approaches provide future opportunities to discover more informative DNA markers over an extensive range.

The development of molecular markers in the biofuel tree Pongamia opens the possibility for further crop improvement and domestication. These processes are slow and are especially hindered in a tree crop where key phenotypic traits, such as oil content or seed yield, are only expressed in a mature form. Finding molecular markers, which can be easily assessed at a juvenile stage, combined with low level (6%) precocious flowering as we observed in Pongamia (see FIG. 6), permits the generation of hybrid material of elite selected tree lines. This can form the basis for further breeding by hybridization, or clonal propagation using either organ culture, grafting or root cuttings. Such association mapping and development of molecular linkage maps for both single gene traits and QTLs are now possible in the near future. PISSR regions are likely to yield conventional SSR markers for clearer and faster association to traits. Together, these molecular genetic approaches will help advance the biotechnological improvement of Pongamia pinnata.

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

### REFERENCES

Agarwal, M., Shrivastava, M. and Padh, H. (2008) Advances in molecular marker techniques and their applications in plant sciences. Plant Cell Rep. 27, 617-631.
Azam, M. M., Waris, A., Nahar, N. M. (2005) Prospects and potential of fatty acid methyl esters of some non-traditional seed oils for use as biodiesel in India. Biomass and Bioenergy, 29, 293-302.
Altschul, S. F., Gish, W., Miller, W., Myers, E. W. and Lipman, D. J. (1990) Basic local alignment search tool. J. Mol. Biol. 215, 403-410.
Ambus, P., Skiba, U., Butterbach-Bahl, K. and Sutton, M. A. (2011) Reactive nitrogen and greenhouse gas flux interactions in terrestrial ecosystems. Plant Soil, 343, 1-3.
Bassam, B. J., Caetano-Anolles, G. & Gresshoff, P. M. (1991) Fast and sensitive silver staining of DNA in polyacrylamide gels. Anal. Biochem. 196, 80-83.
Bassam, B. J. & Gresshoff, P. M. (2007). Silver staining DNA in polyacrylamide gels. Nature Protocols, 2, 2649-2654.
Biswas, B., Scott, P. T. and Gresshoff, P. M. (2011) Tree Legumes as Feedstock for Sustainable Biofuel Production: Opportunities and Challenges. Journal of Plant Physiology, available online: doi:10.1016/j.jplph.2011.05.015.
Caetano-Anolles, G., Bassam, B. J. & Gresshoff, P. M. (1991) DNA amplification fingerprinting using very short arbitrary oligonucleotide primers. BioTechnology, 9, 553-557.
Christie, W. W. (1993) Advances in Lipid Methodology—Two, pp. 195-213
Cobos, M. J., Winter, P., Kharrat, M., Cubero, J. I., Gil, J., Millan, T. and Rubio, J. (2009) Genetic analysis of agronomic traits in a wide cross of chickpea. Field Crops Res. 111, 130-136.
Crutzen, P. J., Mosier, A. R., Smith, K. A. and Winiwarter, W. (2007) N₂O release from agro-biofuel production negates global warming reduction by replacing fossil fuels. Atmos. Chem. Phys. Discuss. 7, 11191-11205.
CSIRO (2011) Flight path to sustainable aviation. Commonwealth Scientific and Industrial Research Organisation (CSIRO).
Doyle, J. J. and Doyle, J. L. (1987) A rapid DNA isolation procedure for small quantities of fresh leaf tissue. Phytochemical Bulletin, 19, 11-15.
El Aabidine, A. Z., Charafi, J., Grout, C., Doligez, A., Santoni, S., Moukhli, A., Jay-Allemand, C., El Modafar, C. and Khaadari, B. (2010) Construction of a genetic linkage map for the olive based on AFLP and SSR markers. Crop Sci. 50, 2291-2302.
Fransen. T., Bhatia, P. and Hsu, A. (2007) The Greenhouse Gas Protocol, Measuring to Manage: A Guide to Designing GHS Accounting and Reporting Programs—Full report 2007. World Resource Institutes www.ghgprotocol.org/files/measuring-to-manage.pdf
Gonzalez, A., Wong, A., Delgado-Salinas, A., Papa, R. and Grepts, P. (2005) Assessment of Inter Simple Sequence Repeat Markers to differentiate sympatric wild and domesticated populations of common bean. Crop Science Society of America, 45, 606-615.
Graham, P. W., Reedman, L. J., Rodrigues, L., Raison, J., Braid, A., Haritos, V., Adams, P., Brinsmead, T. S., Hayward, J. A., Taylor, J. and O'Connell, D. (2011) Sustainable aviation fuels roadmap; data assumptions and modeling, Commonwealth Scientific and Industrial Research Organisation (CSIRO).
Gupta, P. K. and Varshney, R. K. (2000) The development and use of microsatellite markers for genetic analysis and plant breeding with emphasis on bread wheat. Euphytica, 113, 163-185.
Hill, J., Nelson, E., Tilman, D., Polasky, S., and Tiffany, D. (2006) Environmental, economic, and energetic costs and benefits of biodiesel and ethanol biofuels. Proc. Nat. Acad. Sci. 103, 11206-11210.
Jensen, E. S., Peoples, M. B., Boddey, R. M., Gresshoff, P. M., Hauggaard-Nielsen, H., Alves, B. J. R., and Morrison, M. J. (2011) Legumes for mitigation of climate change and feedstock in a bio-based economy—A review, Agricultural Development for Sustainability (in press).

Kazakoff, S. H., Gresshoff, P. M., Scott, P. T. (2011) Pongamia pinnata, a sustainable feedstock for biodiesel production. In: Halford N G, Karp A, editors. Energy Crops, Cambridge, UK: Royal Society for Chemistry, p. 233-258.
Karmee, S. K. and Chadha, A. (2005) Preparation of biodiesel from crude oil from Pongamia pinnata. Bioresources Tech. 96, 1425-1429.
Kesari, V, Sathyanarayana, V. M., Parida, A. and Rangan, L. (2010) Molecular marker-based characterization in candidate plus trees of Pongamia pinnata, a potential biodiesel legume, AoB Plants 2010: plq017.
Lin, K. H., Yeh, W. L., Chen H. M. and Lo, H. F. (2010) Quantitative trait loci influencing fruit-related characteristics of tomato grown in high-temperature conditions. Euphytica, 174, 119-135.
Marshall, D. J., Hayward, A., Eales, D., Imelfort, M., Stiller, J., Berkman, P. J., Clark, T., McKenzie, M., Lai, K., Duran, C., Jacqueline Batley, J., and Edwards D. (2010) Targeted identification of genomic regions using TAGdb. PLANT METHODS, 6, 19. doi:10.1186/1746-4811-6-19.
Meher, L. C., Vidya, S. D., Naik, S. N. (2006) Optimization of Alkali-catalyzed transesterrefication of Pongamia pinnata oil for production of biodiesel. Bioresources Tech. 97, 1392-1397.
Murray, M. G., Thompson, W. F. (1980) Rapid isolation of high molecular weight plant DNA. Nucleic Acids Res. 8, 4321-4325.
Odeh, I. O. A., Tan, D. K. Y., and Ancev, T. (2011) Potential suitability and viability of selected biodiesel crops in Australian marginal agricultural lands under current and future climates. Bioenergy Research, 4, 167-179.
Pérez-Vega, E., Pañeda, A., Rodríguez-Suaréz, C., Campa, A., Giraldez, R. and Ferreira, J. J. (2010) Mapping of QTLs for morpho-agronomic and seed quality traits in a RIL population of common bean (Phaseolus vulgaris L.). Theor. Appl. Genet. 120, 1367-1380.
Petersen, J. R., Okorodudu, A. O., Mohammad, A., Payne, D. A. (2003) Capillary electrophoresis and its application in the clinical laboratory. Clinica Chimica Acta, 330, 1-30.
Rout, G. R., Sahoo, D. P. and Aparajita, S. (2009) Studies on inter and intra-population variability of Pongamia pinnata: a bioenergy legume tree. Crop Breed. Appl. Biotechnol. 9, 268-273.
Rockström J, Falkenmark M, Karlberg L, Hoff H, Rost S, Gerten D. (2009) Future water availability for global food production: the potential of green water for increasing resilience to global change, Water Resources Research. 45: W00A12. doi:10.1029/2007WR006767.
Scott, P. T., Pregelj, L., Chen, N., Hadler, J. S., Djordjevic, M. A. and Gresshoff, P. M. (2008) Pongamia pinnata: An untapped resource for the biofuels industry of the future. Bioenerg. Res. 1, 2-11.
Sahoo, D. P., Aparajita, S. and Rout, G. R. (2010) Inter and intra-population variability of Pongamia pinnata: a bioenergy legume tree. Plant Syst. Evol. 285, 121-125.
Sharma, S. N., Kumar, V. and Mathur, S. (2009) Comparative analysis of RAPD and ISSR markers for characterization of sesame (Sesamum indicum L) genotypes. J. Plant Biochem. Biotechnol. 18, 37-43.
Sharma, S. S., Negi, M. S., Sinha, P., Kumar, K. and Tripathi, S. B. (2010) Assessment of Genetic Diversity of biodiesel species Pongamia pinnata accessions using AFLP and Three Endonuclease-AFLP. Plant Mol Biol Rep. 29, 12-18.
Schmid, P. (1973) Extraction and purification of lipids: II. Why is chloroform-methanol such a good lipid solvent. Physiol. Chem. Phys. 5, 141-150.
Singh, M., Bandana, Abuja, P. S., (1999) Isolation and PCR amplification of genomic DNA from market samples of dry tea. Plant Molecular Biology Reporter, 17, 171-178.
Stemmer, W. P. C. (2002) Molecular breeding of genes, pathways and genomes by DNA shuffling. Journal of Molecular Catalysis B: Enzymatic, 19-20, 3-12.
Sujatha, K., Rajwade, A. V., Gupta, V. S. and Hazra, L. (2010) Assessment of Pongamia pinnata (L.)—a biodiesel producing tree species using ISSR markers. Curr. Sci. 99, 1327-1329.
Tanya, P., Taeprayoon, P., Hadkam, Y. and Srinives, P. (2011) Genetic diversity among Jatropha and Jatropha-related species based on ISSR markers. Plant Mol. Biol. Rep. 29, 252-264.
Sutton, M. A., Howard, C. M., Erisman, J. W., Billen, G., Bleeker, A., Grennfelt, P., Grinsven, H. V., and Grizzetti, B. (2011) (eds) The European Nitrogen Assessment (Cambridge Univ. Press); available at go.nature.com/5n91sq
Wilkinson, C. S., Fuskhah, E., Indrasumunar, A., Hayashi, S., Gresshoff, P. M., and Scott, P. T. (2011) Growth, nodulation and nitrogen gain of Pongamia pinnata and Glycine max in response to salinity. BioEnergy Research, (In preparation).
Wolfe, A., Xiang, Q., and Kephart, S. (1998) Assessing hybridization in natural populations of Penstemon (Scrophulariaceae) using hypervariable intersimple sequence repeat (ISSR) bands. Molecular Ecology, 7, 1107-1125.
Zietkiewicz, E., Rafalski, A. and Labuda, D. (1994) Genome fingerprinting by simple sequence repeat (SSR)-anchored polymerase chain reaction amplification. Genomics, 20, 176-183.

TABLE 1
Variation of seed mass, seed oil and oleic acid content
in Pongamia trees

| | | | % seed | |
|---|---|---|---|---|
| | *sample | Seed mass | oil/seed | % oleic |
| No. | ID | (g) | mass | acid/seed oil |
| 1 | T10-6 | 1.34 | 50.5 | 51.6 |
| 2 | T11 | 1.5 | 33.2 | 47.9 |
| 3 | N90 | 1.37 | 32.6 | 39.9 |
| 4 | G32-2 | 1.31 | 35.3 | 43.8 |
| 5 | OT1 | 0.62 | 33.4 | 25.4 |
| 6 | GC1 | 0.41 | 33.3 | 43.9 |
| 7 | GC2 | 0.61 | 42.0 | 54.2 |
| 8 | GC3 | 0.75 | 37.1 | 38.8 |
| 9 | GT1-30 | 1.02 | 19.7 | 45.6 |
| 10 | GT2-164 | 0.92 | 35.7 | 39.2 |
| | AVE ± STD | 0.99 ± 0.4 | 35.3 ± 7.8 | 43.1 ± 8.1 |

*The seed samples were collected from Taringa, Milton, Gatton and Ascot (Brisbane, QLD) in December 2010.

TABLE 2
Variation of seed oil and oleic acid in progeny from a single tree

| | | | % seed | |
|---|---|---|---|---|
| | sample | Seed mass | oil/seed | % oleic |
| No. | ID | (g) | mass | acid/seed oil |
| 1 | T10-1 | 0.97 | 46.4 | 51.7 |
| 2 | T10-2 | 1.07 | 52.3 | 60.8 |
| 3 | T10-3 | 1.29 | 40.3 | 53.2 |
| 4 | T10-4 | 1.34 | 42.5 | 56.9 |
| 5 | T10-5 | 1.37 | 47.4 | 68.3 |
| 6 | T10-6 | 1.34 | 50.5 | 51.6 |
| | AVE ± STD | 1.2 ± 0.2 | 46.6 ± 4.4 | 57.2 ± 6.5 |

T10-1 to T10-6 are single seeds derived from mother tree T10.

TABLE 3
Nucleotide sequence of PISSR primers

| | Primer sequence | |
|---|---|---|
| PISSR primer | 5′-> 3′ | SEQ ID NO |
| PISSR1 | (GA)₈AT | 1 |
| PISSR2 | (GA)₈AA | 2 |
| PISSR3 | (GA)₈CG | 3 |
| PISSR4 | (GA)₈TG | 4 |
| PISSR5 | (GA)₈TA | 5 |
| PISSR6 | (GA)₈CA | 6 |
| PISSR7 | CA(GA)₈ | 185 |
| PISSR8 | GT(GA)₈ | 186 |
| PISSR9 | AA(GA)₈ | 187 |
| PISSR10 | TC(GA)₈ | 188 |
| PISSR11 | TA(GA)₈ | 189 |
| PISSR12 | AG(GA)₈ | 190 |
| PISSR13 | (CA)₈AAC | 7 |
| PISSR14 | (CA)₈ATG | 8 |
| PISSR15 | (CA)₈AGA | 9 |
| PISSR16 | (CA)₈ACT | 10 |
| PISSR17 | (CA)₈TAG | 11 |
| PISSR18 | (CA)₈ATT | 12 |
| PISSR19 | (CA)₈TGC | 13 |
| PISSR20 | (CA)₈TCA | 14 |
| PISSR21 | (CA)₈GAG | 15 |
| PISSR22 | (CA)₈GTC | 16 |
| PISSR23 | (CA)₈GGT | 17 |
| PISSR24 | (CA)₈GCA | 18 |
| PISSR25 | (CA)₈CTC | 19 |
| PISSR26 | (CA)₈CGA | 20 |
| PISSR27 | (CA)₈CCT | 21 |
| PISSR 28 | (AT)₈TTA | 22 |
| PISSR 29 | (AT)₈TAT | 23 |
| PISSR 30 | (AT)₈TGG | 24 |
| PISSR 31 | (AT)₈TCA | 25 |
| PISSR 32 | (AT)₈GGC | 26 |
| PISSR 33 | (AT)₈GTA | 27 |
| PISSR 34 | (AT)₈GAG | 28 |
| PISSR 35 | (AT)₈GCT | 29 |
| PISSR 36 | (AT)₈AAC | 30 |
| PISSR 37 | (AT)₈ACG | 31 |
| PISSR 38 | (AT)₈AGG | 32 |
| PISSR 39 | (AT)₈CTA | 33 |
| PISSR 40 | (AT)₈CCG | 34 |
| PISSR 41 | (AT)₈CAC | 35 |
| PISSR 42 | (AT)₈CGT | 36 |
| PISSR 43 | (CT)₈AAT | 37 |
| PISSR 44 | (CT)₈ATA | 38 |
| PISSR 45 | (CT)₈ACG | 39 |
| PISSR 46 | (CT)₈AGC | 40 |
| PISSR 47 | (CT)₈TAA | 41 |
| PISSR 48 | (CT)₈TTG | 42 |
| PISSR 49 | (CT)₈TCT | 43 |
| PISSR 50 | (CT)₈TGG | 44 |
| PISSR 51 | (CT)₈CAG | 45 |
| PISSR 52 | (CT)₈CCT | 46 |
| PISSR 53 | (CT)₈CGG | 47 |
| PISSR 54 | (CT)₈GAC | 48 |
| PISSR 55 | (CT)₈GTG | 49 |
| PISSR 56 | (CT)₈GCT | 50 |
| PISSR 57 | (CT)₈GGC | 51 |
| PISSR 58 | (CT)₈AAA | 52 |
| PISSR 59 | (CT)₈AAC | 53 |
| PISSR 60 | (CT)₈AAG | 54 |
| PISSR 61 | (CT)₈ATT | 55 |
| PISSR 62 | (CT)₈ATC | 56 |
| PISSR 63 | (CT)₈ATG | 57 |
| PISSR 64 | (CT)₈ACA | 58 |
| PISSR 65 | (CT)₈ACT | 59 |
| PISSR 66 | (CT)₈ACC | 60 |
| PISSR 67 | (CT)₈AGA | 61 |
| PISSR 68 | (CT)₈AGT | 62 |
| PISSR 69 | (CT)₈AGG | 63 |
| PISSR 70 | (CT)₈TAT | 64 |
| PISSR 71 | (CT)₈TAC | 65 |
| PISSR 72 | (CT)₈TAG | 66 |
| PISSR 73 | (CT)₈TTA | 67 |
| PISSR 74 | (CT)₈TTT | 68 |
| PISSR 75 | (CT)₈TTC | 69 |
| PISSR 76 | (CT)₈TCA | 70 |
| PISSR 77 | (CT)₈TCC | 71 |
| PISSR 78 | (CT)₈TCG | 72 |
| PISSR 79 | (CT)₈TGA | 73 |
| PISSR 80 | (CT)₈TGT | 74 |
| PISSR 81 | (CT)₈TGC | 75 |
| PISSR 82 | (CT)₈CAA | 76 |
| PISSR 83 | (CT)₈CAT | 77 |
| PISSR 84 | (CT)₈CAC | 78 |
| PISSR 85 | (CT)₈CTT | 79 |
| PISSR 86 | (CT)₈CTA | 80 |
| PISSR 87 | (CT)₈CTC | 81 |
| PISSR 88 | (CT)₈CTG | 82 |
| PISSR 89 | (CT)₈CCA | 83 |
| PISSR 90 | (CT)₈CCC | 84 |
| PISSR 91 | (CT)₈CCG | 85 |
| PISSR 92 | (CT)₈CGA | 86 |
| PISSR 93 | (CT)₈CGT | 87 |
| PISSR 94 | (CT)₈CGC | 88 |
| PISSR 95 | (CT)₈GAA | 89 |
| PISSR 96 | (CT)₈GAT | 90 |
| PISSR 97 | (CT)₈GAG | 91 |
| PISSR 98 | (CT)₈GTA | 92 |
| PISSR 99 | (CT)₈GTT | 93 |
| PISSR 100 | (CT)₈GTC | 94 |
| PISSR 101 | (CT)₈GCA | 95 |
| PISSR 102 | (CT)₈GCC | 96 |
| PISSR 103 | (CT)₈GCG | 97 |
| PISSR 104 | (CT)₈GGA | 98 |
| PISSR 105 | (CT)₈GGT | 99 |
| PISSR 106 | (CT)₈GGG | 100 |
| PISSR 107 | (AT)₈TTC | 101 |
| PISSR 108 | (AT)₈TTG | 102 |
| PISSR 109 | (AT)₈TTT | 103 |
| PISSR 110 | (AT)₈TAG | 104 |
| PISSR 111 | (AT)₈TAA | 105 |
| PISSR 112 | (AT)₈TAC | 106 |
| PISSR 113 | (AT)₈TGA | 107 |
| PISSR 114 | (AT)₈TGT | 108 |
| PISSR 115 | (AT)₈TGC | 109 |
| PISSR 116 | (AT)₈TCT | 110 |
| PISSR 117 | (AT)₈TCC | 111 |
| PISSR 118 | (AT)₈TCG | 112 |
| PISSR 119 | (AT)₈GGG | 113 |
| PISSR 120 | (AT)₈GGA | 114 |
| PISSR 121 | (AT)₈GGT | 115 |
| PISSR 122 | (AT)₈GTG | 116 |
| PISSR 123 | (AT)₈GTT | 117 |
| PISSR 124 | (AT)₈GTC | 118 |
| PISSR 125 | (AT)₈GAA | 119 |
| PISSR 126 | (AT)₈GAC | 120 |
| PISSR 127 | (AT)₈GAT | 121 |
| PISSR 128 | (AT)₈GCA | 122 |
| PISSR 129 | (AT)₈GCC | 123 |
| PISSR 130 | (AT)₈GCG | 124 |
| PISSR 131 | (AT)₈ATA | 125 |
| PISSR 132 | (AT)₈ATT | 126 |
| PISSR 133 | (AT)₈ATG | 127 |
| PISSR 134 | (AT)₈AAA | 128 |
| PISSR 135 | (AT)₈AAG | 129 |
| PISSR 136 | (AT)₈AAT | 130 |
| PISSR 137 | (AT)₈ACA | 131 |
| PISSR 138 | (AT)₈ACC | 132 |
| PISSR 139 | (AT)₈ACT | 133 |
| PISSR 140 | (AT)₈AGA | 134 |
| PISSR 141 | (AT)₈AGC | 135 |
| PISSR 142 | (AT)₈AGT | 136 |
| PISSR 143 | (AT)₈CTG | 137 |
| PISSR 144 | (AT)₈CTC | 138 |
| PISSR 145 | (AT)₈CTT | 139 |
| PISSR 146 | (AT)₈CCC | 140 |
| PISSR 147 | (AT)₈CCT | 141 |
| PISSR 148 | (AT)₈CCA | 142 |
| PISSR 149 | (AT)₈CAA | 143 |
| PISSR 150 | (AT)₈CAT | 144 |
| PISSR 151 | (AT)₈CAG | 145 |
| PISSR 152 | (AT)₈CGG | 146 |
| PISSR 153 | (AT)₈CGA | 147 |
| PISSR 154 | (AT)₈CGC | 148 |

TABLE 4
Selected PISSR primers used for DNA marker analysis by PAGE/SS

| | | Total | Range of | Number of |
|---|---|---|---|---|
| | | number | marker sizes | polymorphic |
| Primer | Primer sequence | of bands | (bp) | markers* |
| PISSR1 | 5′(GAGAGAGAGAGAGAGA) | 20-23 | 250-1600 | 15 |
| | AT3′ | | | |
| PISSR2 | 5′(GAGAGAGAGAGAGAGA) | 18-21 | 300-1700 | 12 |
| | AA3′ | | | |
| PISSR3 | 5′(GAGAGAGAGAGAGAGA) | 16-22 | 400-1850 | 11 |
| | CG3′ | | | |
| PISSR4 | 5′(GAGAGAGAGAGAGAGA) | 16-21 | 350-1800 | 11 |
| | TG3′ | | | |
| PISSR5 | 5′(GAGAGAGAGAGAGAGA) | 13-16 | 350-1700 | 10 |
| | TA3′ | | | |
| PISSR6 | 5′(GAGAGAGAGAGAGAGA) | 18-21 | 600-1700 | 9 |
| | GA3′ | | | |
| PISSR7 | 5′CA(GAGAGAGAGAGAGA | 15-19 | 300-1650 | 0 |
| | GA)3′ | | | |
| PISSR8 | 5′GT(GAGAGAGAGAGAGA | 17-21 | 400-1900 | 0 |
| | GA)3′ | | | |
| PISSR13 | 5′(CACACACACACACACA) | 15-17 | 550-1700 | 7 |
| | AAC3′ | | | |
| PISSR14 | 5′(CACACACACACACACA) | 17-19 | 400-1900 | 8 |
| | ATG3′ | | | |
| PISSR17 | 5′(CACACACACACACACA)T | 10-15 | 650-1700 | 9 |
| | AG3′ | | | |
| PISSR18 | 5′(CACACACACACACACA) | 17-20 | 700-1700 | 13 |
| | ATT3′ | | | |

*Total number of polymorphic markers generated from 29 Pongamia samples

TABLE 5
Selected PISSR primers used for DNA marker
analysis by CE

| | | | | Total |
|---|---|---|---|---|
| | | Range | Maximal | number |
| | | of | number of | of |
| | | marker | markers | poly- |
| | | sizes | in one | morphic |
| Primer | Sequence | (bp) | sample | markers* |
| PISSR 14 | 5′(CA)8ATG3′ | 86-396 | 13 | 51 |
| PISSR 17 | 5′(CA)8TAG3′ | 80-374 | 14 | 24 |
| PISSR 18 | 5′(CA)8ATT3′ | 81-371 | 14 | 16 |
| PISSR 20 | 5′(CA)8TCA3′ | 82-396 | 15 | 49 |
| PISSR 22 | 5′(CA)8GTC3′ | 87-386 | 29 | 53 |
| PISSR 24 | 5′(CA)8GCA3′ | 82-393 | 12 | 22 |
| PISSR 25 | 5′(CA)8CTC3′ | 81-398 | 14 | 35 |
| PISSR 26 | 5′(CA)8CGA3′ | 81-394 | 11 | 26 |

*Total number of polymorphic markers generated from 22 Pongamia samples

TABLE 6
Nucleotide sequence relatedness of cloned PISSR markers
extracted from PAGE/SS gels to three model legumes

| | | | | | Soybean | | | |
|---|---|---|---|---|---|---|---|---|
| | | | Lotus japonicus | | (Glycine max) | | Medicago truncatula | |
| | | HSP (high scoring segment pairs) | | | | | | |
| | | Length | Length | | Length | | Length | |
| Primer | Marker | (bp) | (bp) | Accession | (bp) | Accession | (bp) | Accession |
| PISSR1 | QJ7 | 768 | 482 | BW594779 | | | | |
| PISSR15 | SH41 | 219 | 1237 | TC57311 | 460 | DB985567 | 438 | BQ140302 |
| PISSR16 | SH23 | 1089 | | | 672 | HO760602 | | |
| PISSR17 | SH21 | 1028 | | | 1476 | TC486715 | 1139 | ES466846 |
| | SH22 | 1054 | | | 672 | HO760602 | 1229 | EC366194 |
| PISSR18 | SH19 | 565 | 240 | GE117618 | | | | |
| PISSR19 | SH24 | 955 | | | 1477 | TC486716 | 1230 | EC366194 |
| | SH34 | 478 | 260 | G0034876 | 241 | FG993806 | 254 | EX528031 |
| PISSR20 | SH12 | 770 | | | 175 | GD716163 | 819 | ES613526 |
| | SH49 | 135 | 712 | TC62522 | 820 | TC438781 | | |
| PISSR21 | SH50 | 184 | 475 | FS324643 | 742 | TC437835 | 1024 | TC189785 |
| PISSR24 | SH53 | 347 | | | | | 668 | TC182451 |
| PISSR26 | SH38 | 184 | 588 | TC78985 | 340 | TC482151 | 532 | TC188978 |
| PISSR27 | SH40 | 224 | | | 549 | TC435357 | | |

*Nucleotide similarities results via BLAST in NCBI/GenBank and DFCI gene indices databases.

TABLE 7
Predicted information content of selected PISSR markers

| | | Lotus | Soybean (Glycine | Medicago |
|---|---|---|---|---|
| Primer | Marker | japonicus | max) | truncatula |
| PISSR1 | QJ7 | MAP kinase | | |
| PISSR15 | SH41 | predicted | predicted protein | predicted protein |
| | | protein | | |
| PISSR16 | SH23 | | nucleic acid binding | |
| PISSR17 | SH21 | | Repetitive proline- | DNA binding |
| | | | rich cell wall protein 1 | |
| | SH22 | | Phenylalanine | single stranded |
| | | | ammonia-lyase 2 | nucleic acid |
| | | | | binding R3H |
| PISSR18 | SH19 | | Hydrolase activity | |
| PISSR19 | SH24 | | Repetitive proline- | Probable histone |
| | | | rich cell wall protein 1 | H2B.1 |
| | SH34 | Transmembrane | predicted protein | cDNA clone |
| | | transport | | |
| PISSR20 | SH12 | | | ATP binding & |
| | | | | ATPase activity |
| | SH49 | RNA binding | ATP binding & | |
| | | | receptor activity | |
| PISSR21 | SH50 | β-amylase | Hairpin inducing | antibody |
| | | activity & cation | activity | |
| | | binding | | |
| PISSR24 | SH53 | | | Cytochrome-c |
| | | | | oxidase |
| | | | | subunit 1 |
| PISSR26 | SH38 | | NAD binding | L-malate |
| | | | | dehydrogenase |
| PISSR27 | SH40 | | ATP binding & | |
| | | | Receptor | |

*Most accession of functional similarities via UniProtKB/SwissProt database (www.uniprot.org)

1. A method of producing an isolated nucleic acid suitable for nucleic acid sequence amplification, said method including the steps of: identifying a genomic nucleotide sequence of a plant of the genus Pongamia according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4; and producing an isolated nucleic acid comprising said nucleotide sequence.

2. An isolated nucleic acid suitable for nucleic acid sequence amplification, said isolated nucleic acid comprising a genomic nucleotide sequence of a plant of the genus Pongamia according to 5′-(Nₓ)_{y}(N)_{z}-3′ wherein each N is the same or different nucleotide and wherein x=2, 3 or 4; y=5, 6, 7, 8, 9 or 10; z=1, 2, 3 or 4.

3. The method of claim 1, wherein x=2 or 3.

4. The method of claim 1, wherein y=8.

5. The method of claim 1, wherein z=2 or 3.

6. The method of claim 1, wherein the isolated nucleic acid comprises a nucleotide sequence according to 5′-(N₂)₈(N)₂-3′.

7. The method of claim 1, wherein Nₓ is CA, AT, CT or GA.

8. The method of claim 1, wherein (N)z is as set forth in any one of SEQ ID NOS:1-148.

9. The method of claim 1, wherein (N)_{z} consists of N₁ and N₂ or N₁, N₂ and N₃, with the proviso that N₂ is a different nucleotide than a second nucleotide of repeat unit (Nₓ)_{y}.

10. The method of claim 1, wherein (Nₓ)_{y} comprises one or more additional same or different nucleotides M that are not repeated, or are repeated to a value less than y.

11. The method of claim 1, wherein the isolated nucleic acid comprises a nucleotide sequence set forth in SEQ ID NOS:1-148.

12. The method of claim 1, wherein the isolated nucleic acid is a PCR primer.

13. A method of genetic analysis including the step of using an isolated nucleic acid according to claim 2, to amplify a plurality of amplification products from a nucleic acid sample obtainable from a plant of the genus Pongamia.

14. (canceled)

15. The method of claim 13, wherein the isolated nucleic acid comprises a nucleotide sequence set forth in SEQ ID NOS:1-148.

16. An isolated nucleic acid comprising a nucleotide sequence set forth in any one of SEQ ID NOS:149-184, or a fragment or variant thereof.

17. A method of genetic analysis including the step of using one or more primers comprising respective nucleotide sequences of an isolated nucleic acid according to claim 16, to amplify one or more amplification products from a nucleic acid sample obtainable from a plant of the genus Pongamia.

18. The method of claim 17, further comprising the step of detecting the one or more amplification products by probe hybridization.

19. (canceled)

20. A method of breeding a plant of the genus Pongamia, said method including the step of producing a progeny plant having a desired trait from one or more parent Pongamia plants, wherein at least one of the parent Pongamia plants is selected as having the desired trait by genetic analysis according to the method of claim 13.

21. The method of claim 20, wherein the desired trait is or relates to seed size, seed number, seed oil content, seed oil quality, seed flavour and toxicity, disease resistance, water use efficiency, nitrogen use efficiency, precocious flowering, flowering time, tree size, tree shape, growth rate, drought tolerance, salinity tolerance and/or growth in low-nutrient soils.

22. (canceled)
